(19) United States
(12) Patent Application Publication
(10) Pub. No.: US 2026/0157732 A1
(43) Pub. Date: Jun. 11, 2026

(54) Apparatus, Method, and System for Medical Imaging and Characterization of Tissue Structure

(71) Applicant: University of Rochester, Rochester, NY (US)

(72) Inventors: Diane Dalecki, Rochester, NY (US); Denise Hocking, Rochester, NY (US); Sarah Wayson, Rochester, NY (US); Maria Helguera, Rochester, NY (US); Nicole Wilson, Rochester, NY (US)

(21) Appl. No.: 19/381,766

(22) Filed: Nov. 6, 2025

Related U.S. Application Data
(60) Provisional application No. 63/716,900, filed on Nov. 6, 2024.

(51) Int. Cl. A61B 8/00; A61B 8/08

(52) U.S. Cl. CPC A61B 8/5223; A61B 8/0858; A61B 8/4483; A61B 8/461; A61B 8/485

(57) ABSTRACT

A method, system, and apparatus for characterizing a tissue is described. The method, system, and apparatus may include steps or operations including positioning ultrasound transducers and ultrasound receivers; insonating a tissue with an ultrasound transducer from at least one position; collecting reflected ultrasound energy from at least one position; and determining at least one characteristic of the tissue based on the collected ultrasound energy.

### CROSS-REFERENCE TO RELATED APPLICATIONS

This application claims priority to U.S. Provisional Application No. 63/716,900 filed on Nov. 6, 2024 incorporated herein by reference in its entirety.

### FEDERALLY SPONSORED RESEARCH AND DEVELOPMENT

This invention was made with government support under GM131281 awarded by the National Institutes of Health. The government has certain rights in the invention.

### BACKGROUND OF THE INVENTION

Alterations to tissue microstructure, resulting from, for example, disease, aging, or injury, can impact tissue function, affect healing from injury or surgery, and influence quality of life. For example, tendinopathy, neuropathy, and injury can each limit range of motion, affect tendon/nerve function, cause pain, and often require surgical repair. One challenge facing clinicians and physical therapists is developing rehabilitation protocols during healing without re-rupturing the repair site. This challenge can be complicated further in patients with diabetes or in aged patients, where pathological changes in tissue microstructure and mechanical properties may occur.

Tendon disorders, including chronic degeneration and acute rupture, limit range of motion, reduce tendon strength, affect tendon function, cause pain, and often require surgical repair. One challenge facing clinicians and physical therapists is developing rehabilitation protocols for tendon during healing without re-rupturing the repair site. This challenge can be complicated further in patients with diabetes, where tendon structure and mechanical properties are impaired (Batista, F. et al. Foot Ankle Int 29, 498-501 (2008), Boivin, G. P. et al. Muscles Ligaments Tendons J 4, 280-284 (2014), Studentsova, V. et al. Sci Rep 8, 9218 (2018), Guney, A. et al. Exp Clin Endocrinol Diabetes 123, 428-432 (2015)). A key structural property of tendon that impacts its mechanical properties is collagen fiber alignment (Lake, S. P. et al. J Orthop Res 27, 1596-1602 (2009)). Collagen fibers are organized along the longitudinal axis of tendon, and they serve as load bearing structures during applied uniaxial tensile loading (Butler, D. L. et al. J Biomech 17, 579-596 (1984)). Thus, developing point-of-care ultrasound imaging techniques that quantitatively characterize tendon collagen fiber microstructure would provide a valuable clinical tool to longitudinally monitor tendon during healing and guide rehabilitation.

Globally, 537 million adults have been diagnosed with diabetes, and the prevalence of diabetes is expected to reach 783 million by 2045 (Federation, I. D. IDF Diabetes Atlas (2021)). Diabetes increases the likelihood of tendon injury and impairs tendon structure, mechanical properties, and healing (Batista, F. et al. Foot Ankle Int 29, 498-501 (2008), Boivin, G. P. et al. Muscles Ligaments Tendons J 4, 280-284 (2014), Studentsova, V. et al. Sci Rep 8, 9218 (2018), Guney, A. et al. Exp Clin Endocrinol Diabetes 123, 428-432 (2015), Ranger, T. A. et al. Br J Sports Med 50, 982-989 (2016), David, M. A. et al. PLOS One 9, e91234 (2014), Ackerman, J. E. et al. PLOS One 12, e0181127 (2017)). Tendons of diabetic patients exhibit collagen fiber disorganization, and diabetic patients are over three times more likely to develop tendinopathy than non-diabetic patients (Batista, F. et al. Foot Ankle Int 29, 498-501 (2008), Boivin, G. P. et al. Muscles Ligaments Tendons J 4, 280-284 (2014), Studentsova, V. et al. Sci Rep 8, 9218 (2018), Ranger, T. A. et al. Br J Sports Med 50, 982-989 (2016)). Furthermore, even if normal metabolic function is restored in diabetic mice, tendinopathy remains, highlighting the potential value of monitoring diabetic tendon structure under effective glycemic management (Studentsova, V. et al. Sci Rep 8, 9218 (2018)).

Similarly, aging disrupts collagen microstructure in tendon, impairing mechanical properties and healing (Korcari, A. et al. Connect Tissue Res, 1-13 (2022)). Tendons accumulate microdamage over time and aging alters collagen fiber alignment (Ackerman, J. E. et al. J Orthop Res (2017)). The chance of tendon rupture also increases with age, and tendon healing is impaired in aged mice, leading to reduced mechanical properties following injury (Ackerman, J. E. et al. J Orthop Res (2017), de Jonge, S. et al. Br J Sports Med 45, 1026-1028 (2011)). Thus, alterations in collagen microstructure, impaired mechanical properties, and diminished healing observed in diabetic and aged tendon highlight the need for tailoring and monitoring therapies to maintain tendon health and promote recovery.

Ultrasound imaging of tendon has typically focused on interpreting B-mode images, and developing semi-quantitative image processing approaches to describe tendon morphology under normal conditions, in the presence of tendinopathy, diabetes, lesions, and during healing (Batista, F. et al., Foot Ankle Int 29, 498-501 (2008), Hullfish, T. J. & Baxter, J. R., J Ultrasound Med (2018), Bashford, G. R., et al. IEEE Trans Med Imaging 27, 608-615 (2008), Docking, S. I. & Cook, J., Scand J Med Sci Sports 26, 675-683 (2016), Duenwald-Kuehl, S., et al. ORS 2012 Annual Meeting, Poster No. 1288 (2012), Hagan, K. L., et al. J Appl Physiol (1985) (2018), Kulig, K., et al. Ultrasound Med Biol 42, 664-673 (2016), Tucker, J. J. et al. J Orthop Res 34, 161-166 (2016), van Schie, H. T. M. et al. Brit J Sport Med 44, 1153-1159 (2010), Abate, M., et al. Foot Ankle Int 35, 44-49 (2014), Akturk, M., et al. Exp Clin Endocrinol Diabetes 115, 92-96 (2007), Couppe, C. et al. J Appl Physiol (1985) 120, 130-137 (2016), de Jonge, S. et al., Br J Sports Med 49, 995-999 (2015), Crevier-Denoix, N. et al. J Biomech 38, 2212-2220 (2005), Riggin, C. N., et al. J Biomech Eng 136, 021029 (2014), Tuthill, T. A., Ultrasound in Medicine and Biology 25, 959-968 (1999), Buschmann, J. et al. Connect Tissue Res 55, 123-131 (2014), Chamberlain, C. S. et al. Ann Biomed Eng 41, 477-487 (2013), Freedman, B. R. et al. J Orthop Res 34, 2172-2180 (2016), Freedman, B. R. et al. J Am Acad Orthop Surg 25, 635-647 (2017), Fryhofer, G. W. et al. J Appl Physiol (1985) 121, 1106-1114 (2016), Ghorayeb, S. R. et al. IEEE Trans Ultrason Ferroelectr Freq Control 59, 694-702 (2012)). There have been efforts to detect tendon damage during loading using B-mode imaging (Duenwald-Kuehl, S., et al., J Biomech 45, 1607-1611 (2012)). There have been efforts to develop ultrasound markers measured from B-mode images that correlate with tendon function (Ackerman, J. S., et al. (2018), Duenwald, S., et al. J Biomech 44, 424-429 (2011), Duenwald-Kuehl, S., et al. J Biomech Eng 134, 111006 (2012), Frisch, K. E., et al. J Biomech 47, 3813-3819 (2014), Lee, S. Y. et al. Sci Rep 7, 5100 (2017), Suydam, S. M. & Buchanan, T. S. J Biomech 47, 1806-1809 (2014)).

An important limitation of B-mode imaging is that it produces qualitative rather than quantitative images. The appearance of B-mode images is impacted by acoustic attenuation, image acquisition system settings, including receive gain, dynamic range, and transducer angle (Cobbold, R. S. C. in Foundations of Biomedical Ultrasound Ch. 7, 414-422 (Oxford University Press, 2007), Szabo, T. L. in Diagnostic Ultrasound Imaging: Inside Out Ch. 10, 387-388 (Elsevier Inc. Academic Press, 2014)). Developing a quantitative ultrasound imaging technique that accounts for the transfer function of the receive system and acoustic attenuation is critical for revealing underlying features of tendon microstructure that are independent of the image acquisition system. Additionally, using high-frequency ultrasound improves imaging resolution and scattering strength compared to current clinical imaging systems that operate with frequencies of approximately 1-15 MHz (Szabo, T. L. in Diagnostic Ultrasound Imaging: Inside Out Ch. 1, 26 (Elsevier Inc. Academic Press, 2014)).

Quantitative ultrasound imaging techniques can provide metrics to characterize native and engineered tissues that are independent of the ultrasound system and user settings. One quantitative ultrasound technique relies upon the integrated backscatter coefficient (IBC) (Oelze, M. L. & Mamou, J. IEEE Trans Ultrason Ferroelectr Freq Control 63, 336-351 (2016), Cloutier, G. et al. Insights Imaging 12, 127 (2021), Dalecki, D. et al. Ann Biomed Eng 44, 636-648 (2016), Mercado, K. P., PHD thesis, University of Rochester, (2015), Mercado, K. P. et al. Ann Biomed Eng 42, 1292-1304 (2014), Mercado, K. P. et al. Tissue Eng Part C Methods 21, 671-682 (2015)). The IBC is a quantitative ultrasound spectral parameter that estimates how strongly scatterers within a resolution cell volume reflect the interrogating ultrasonic pulse back to the transducer. The IBC is the sum of the ratio of backscattered to incident intensity, per unit volume of tissue, over frequencies within the transducer bandwidth (Cobbold, R. S. C. in Foundations of Biomedical Ultrasound Ch. 5, 270-271 (Oxford University Press, 2007), Insana, M. F. & Hall, T. J. Ultrason Imaging 12, 245-267 (1990)). IBC can depend on angle of incidence for phantoms with aligned structure, and tissues with aligned fibers, including myocardium, tendon, and skeletal muscle (Mottley, J. G. & Miller, J. G. et al. J Acoust Soc Am 83, 755-761 (1988), Hoffmeister, B. K., et al. J Acoust Soc Am 97, 1307-1313 (1995), Holland, M. R. et al. J Am Soc Echocardiogr 11, 929-937 (1998), Madaras, E. I., et al. J Acoust Soc Am 83, 762-769 (1988), Hall, C. S., et al. J Acoust Soc Am 107, 612-619 (2000), Wickline, S. A., et al. Circulation 85, 259-268 (1992), Holland, M. R. et al. J Am Soc Echocardiogr 10, 511-517 (1997), Hete, B. & Shung, K. K. IEEE Trans Ultrason Ferroelectr Freq Control 40, 354-365 (1993)). Ultrasound backscatter in tendon has shown an angular dependence of the power spectra of backscattered echoes in healthy equine tendon that was not observed in injured tendon (Garcia, T., et al. Ultrasound Med Biol 29, 1787-1797 (2003)). The IBC as a function of insonation angle was measured and its anisotropy was demonstrated in healthy tendon ex vivo (Hoffmeister, B. K., et al. J Acoust Soc Am 97, 1307-1313 (1995)). No studies have reported usage of the IBC as a function of insonation angle to characterize alterations in tendon organization in the presence of disease or aging.

The integrated backscatter coefficient (IBC) is a quantitative ultrasound metric that provides an estimate of tissue scattering strength and is independent of the image acquisition system (Insana M. F., Hall T J. (1990). Ultrason Imaging 12, 245-267). The IBC is the sum of the ratio of backscattered to incident intensity, per unit volume of tissue, over frequencies within the transducer's detectable bandwidth (Insana M. F., Hall T J. (1990). Ultrason Imaging 12, 245-267).

Thus, there is a need in the art for improved imaging systems that can easily be implemented at the point of care, for characterizing tissue organization and diagnosing and monitoring a variety of conditions.

### SUMMARY OF THE INVENTION

In some aspects, the invention relates to a method of characterizing a tissue.

In some embodiments, the method of characterizing a tissue includes the steps of providing a set of at least one ultrasound transducers and a set of at least one ultrasound receivers, insonating the tissue with one of the set of ultrasound transducers from a first position, collecting a first reflected ultrasound energy with at least one of the set of ultrasound receivers, insonating the tissue with one of the set of ultrasound transducers from a second position, collecting a second reflected ultrasound energy with at least one of the set of ultrasound receivers, calculating a first and second average integrated backscatter coefficient of the tissue from the first and second reflected ultrasound energies respectively, and determining at least one characteristic of the tissue based on the first and second integrated backscatter coefficients.

In some embodiments, at least one of the ultrasound transducers is a single-element transducer, a multi-element transducer, an array transducer, a 1-D array transducer, a 1.5-D array transducer, a 2-D array transducer, a ring array transducer, or a linear array transducer.

In some embodiments, the tissue is a tendon, a ligament, a cartilage, a nerve, a heart, a cornea, a skeletal muscle, a smooth muscle, connective tissues, liver, skin, extracellular matrices, or an artificially fabricated tissue.

In some embodiments, the method includes the step of positioning the transducer automatically.

In some embodiments, the reflected ultrasound energies includes a backscattered echo.

In some embodiments, at least one of the insonation positions is defined by an insonation angle of about 90° relative to the tissue surface.

In some embodiments, the method includes the steps of insonating the tissue with one of the set of ultrasound transducers from a third position, collecting a third reflected ultrasound energy with at least one of the set of ultrasound receivers, and calculating a third average integrated backscatter coefficient of the tissue from the third reflected ultrasound energy.

In some embodiments, the first insonation position comprises an insonation angle less than 90° relative to the tissue surface, the second insonation position comprises an insonation angle of about 90° relative to the tissue surface, and the third insonation position comprises an insonation angle of greater than 90° relative to the tissue surface.

In some embodiments, determining the at least one characteristic of the tissue is further based on average integrated backscatter coefficients from first, second, and third insonation positions.

In some embodiments, the method includes the step of quantifying the relationship between the average integrated backscatter coefficient and the insonation angle of the position.

In some embodiments, quantifying the relationship between the average integrated backscatter coefficient and the insonation angle comprises determining a Gaussian fit of the average integrated backscatter coefficient and the insonation angle.

In some embodiments, quantifying the relationship between estimated integrated backscatter coefficient and the insonation angle comprises calculating one or more metrics in addition to determining a Gaussian fit.

In some embodiments, the calculated metric is the maximum average integrated backscatter coefficient value of the Gaussian fit, the change in amplitude of the Gaussian fit between the maximum value and the value corresponding with any other insonation angle, the linear rate of change of the derivative of the Gaussian fit, or the difference between the first and second average backscatter coefficients.

In some embodiments, the calculated metric is the change in amplitude of the Gaussian fit between the maximum value and the value corresponding with any other insonation angle, and wherein the any other insonation angle is within the range of about 70° to about 110° relative to the tissue surface.

In some embodiments, the characteristic of the tissue is a property of the tissue structure.

In some embodiments, the property of the tissue structure is tissue microstructure, extracellular matrix fiber organization, extracellular matrix fiber alignment, collagen fiber organization, collagen fiber alignment, extracellular matrix fiber crosslinking, collagen fiber crosslinking, distribution of extracellular matrix fiber subtypes, distribution of collagen fiber subtypes, extracellular matrix fiber density, collagen fiber density, extracellular matrix fiber length, collagen fiber length, cellular alignment, cellular organization, vascular alignment, or vascular organization.

In some embodiments, the characteristic of the tissue is a material property of the tissue.

In some embodiments, the material property of the tissue is stiffness, young's modulus, transition strain, transition stress, yield strain, yield stress, ultimate strain, ultimate stress, compressive load, tensional load, or viscoelasticity.

In some embodiments, the characteristic of the tissue is age, inflammation, vascularization, scarring, fibrosis, or steatosis.

In some embodiments, the method includes the step of diagnosing a subject based on the determination of the characteristic of the tissue.

In some embodiments, the diagnosis includes assigning a risk of tissue injury, diagnosing a subject with an autoimmune disease, diagnosing a subject with a metabolic disease, or determining the healing of an injured tissue.

In some embodiments, the method includes the step of determining a treatment for the subject based on the determination of the at least one characteristic of the tissue or determining a rehabilitation therapy for the subject based on the determination of the at least one characteristic of the tissue.

In some aspects, the invention relates to a system for characterizing a tissue. In some embodiments, the system includes a display, a processor communicatively connected to the imaging device and the display, a non-transitory computer readable medium with instructions stored thereon, which when executed by a processor perform steps including: insonating the tissue with one of the set of ultrasound transducers from a first position, collecting a first reflected ultrasound energy with at least one of the set of ultrasound receivers, insonating the tissue with one of the set of ultrasound transducers from a second position, collecting a second reflected ultrasound energy with at least one of the set of ultrasound receivers, calculating a first and second average integrated backscatter coefficient of the tissue from the first and second reflected ultrasound energies respectively, and determining at least one characteristic of the tissue based on the first and second integrated backscatter coefficients.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts an exemplary embodiment of a high-frequency ultrasound image acquisition setup. FIG. 1 depicts murine tails and or other tissues submerged in a tank containing degassed phosphate-buffered saline (PBS). A 58-MHz single element, focused, immersion transducer is depicted scanning along the sagittal plane of the tail. A-lines are acquired in 60-μm steps. The pulse-receiver has a pulse repetition frequency of 200 Hz and gain of 50 dB. Ten backscattered echoes are averaged for each A-line. The platform holding the tissue is rotated to enable imaging at 31 insonification angles in increments of 1°.

FIG. 2A through FIG. 2B depict quantitative ultrasound metrics for characterizing the IBC as a function of insonification angle. FIG. 2A depicts the IBC normalized by the number of fascicle edges (FE) in each ROI, I̅B̅C̅_{N}, plotted as a function of insonification angle. The maximum normalized IBC, I̅B̅C̅_{N,max}, provides a metric of scattering strength. The change I̅B̅C̅_{N} within 10° of I̅B̅C̅_{N,max}, ΔI̅B̅C̅_{N}, provides a metric of the angular dependence of the scattering strength. FIG. 2B depicts the derivative of I̅B̅C̅_{N},

$$\frac{d {\overline{IBC}}_{N}}{d {θ}_{i}} ,$$

plotted as a function of insonification angle. The slope, M, of the linear region of

$$\frac{d {\overline{IBC}}_{N}}{d {θ}_{i}}$$

as a function of angle provides an additional metric of the angular dependence of the IBC near normal incidence.

FIG. 3A through FIG. 3B depict high frequency transducer impulse response and reference power spectrum. FIG. 3A depicts representative acoustic echo from steel reflector. The wavelength in water was 27 μm, and the axial pulse length was 55 μm.

FIG. 3B depicts the reference power spectrum from the steel reflector. The center frequency was 58 MHz, the bandwidth (−6 dB) was 55 MHz, and minimum and maximum frequencies were 31 MHz and 86 MHz, respectively.

FIG. 4A through FIG. 4D depict scatterer classification and B-mode image interpretation for imaging murine tendon at 58-MHz, and IBC estimation as a function of insonification angle for a tail tendon from a young, wild-type, female mouse. FIG. 4A depicts an exemplary embodiment of imaging tendon. The resolution cell volume of the transducer is an ellipsoid with an axial depth of 27 μm and lateral and transaxial widths of 43 μm. Tendon and fascicles are resolvable and produce specular echoes. Fibers at least 27 μm thick are resolvable. Fibers with 10-27 μm diameters are classified as diffractive scatterers. Collagen fibrils, comprised of collagen molecules, are classified as diffuse scatterers. FIG. 4B depicts a sagittal cross-section of a murine tail at normal incidence. FIG. 4C depicts the IBC averaged over an ROI of 1 mm×250 μm (IBC_{ROI}) at insonification angles of 76°-106° for two planes separated by 120 μm as solid lines. Dotted lines are Gaussian fits to the IBC_{ROI} data. FIG. 4D depicts a Gaussian fit of the IBC_{ROI} normalized by number of fascicle edges averaged for two planes, I̅B̅C̅_{N}, as a function of insonification angle for one representative murine tendon.

FIG. 5A through FIG. 5L depict IBC parametric images and IBC as a function of insonification angle for wild-type murine tail tendon, overlying tail skin, and liver. FIG. 5A through FIG. 5C depict IBC parametric ROIs overlaid on B-mode images for murine tail tendon at select insonification angles of 90°, 85°, and 80°. FIG. 5D depicts I̅B̅C̅_{N} as a function of insonation angle in murine tail tendon (n=4). FIG. 5E through FIG. 5G depict IBC parametric ROIs overlaid on B-mode images for murine tail skin at select insonification angles of 90°, 85°, and 80°. FIG. 5H depicts I̅B̅C̅_{ROI} as a function of insonation angle in murine tail skin (n=4). IBC_{ROI} was not dependent upon angle of incidence in skin. FIG. 5I through FIG. 5K depict IBC parametric ROIs overlaid on B-mode images for murine liver at select insonification angles of 90°, 85°, and 80°. FIG. 5L depicts I̅B̅C̅_{ROI} as a function of insonation angle in murine liver (n=4). IBC did not exhibit angular dependence in liver, and had weaker backscatter compared to skin.

FIG. 6A through FIG. 6D depict IBC estimates in wild-type murine tail tendon at intratendon and intrafascicular levels and in wild-type murine tail skin. FIG. 6A depicts a B-mode image and an IBC parametric image in tail tendon with an intratendon ROI of dimensions 1 mm×250 μm. FIG. 6B depicts IBC_{ROI} as a function of insonation angle for ROIs at intratendon and intrafascicular regions, and in skin. The amplitude of IBC_{ROI} was dependent upon angle for intrafascicular and intratendon regions but was independent of angle in skin. Amplitude of IBC_{ROI} was greatest for intratendon regions and lowest in skin. FIG. 6C depicts a B-mode image and an IBC parametric image in tail tendon with an intrafascicular ROI of dimensions 1 mm×55 μm. FIG. 6D depicts a B-mode image and an IBC parametric in tail skin with ROI of dimensions 1 mm×200 μm.

FIG. 7A through FIG. 7I depict IBC parametric images and quantitative IBC metrics for tendons from wild-type, aged, and diabetic mice. FIG. 7A depicts representative B-mode and IBC parametric images of tail tendons from young, wild-type, female mice. FIG. 7B depicts representative B-mode and IBC parametric images of tail tendons from aged, wild-type, female mice. FIG. 7C depicts representative B-mode and IBC parametric images of tail tendons from young, diabetic, female mice. FIG. 7D depicts representative plots of the I̅B̅C̅_{N} as a function of insonation angle for tendons from young wild-type mice. FIG. 7E depicts representative plots of the I̅B̅C̅_{N} as a function of insonation angle for tendons from aged wild-type mice. FIG. 7F depicts representative plots of the I̅B̅C̅_{N} as a function of insonation angle for tendons from young diabetic mice. FIG. 7G depicts I̅B̅C̅_{N,max} data for young, wild-type (blue columns), aged, wild-type (green columns), and young, diabetic (red columns) tendon. Height of each column represents mean value of the metric, and error bars are standard error of the mean. Data points represent data from individual mice (young, wild-type, n=8; aged, wild-type, n=9; young, diabetic, n=7). Significant differences were determined using one-way analysis of variance and Dunnett's multiple comparisons test (*p<0.05). FIG. 7H depicts ΔI̅B̅C̅_{N} data for young, wild-type (blue columns), aged, wild-type (green columns), and young, diabetic (red columns) tendon. Height of each column represents mean value of the metric, and error bars are standard error of the mean. Data points represent data from individual mice (young, wild-type, n=8; aged, wild-type, n=9; young, diabetic, n=7). Significant differences were determined using one-way analysis of variance and Dunnett's multiple comparisons test (*p<0.05). FIG. 7I depicts M data for young, wild-type (blue columns), aged, wild-type (green columns), and young, diabetic (red columns) tendon. Height of each column represents mean value of the metric, and error bars are standard error of the mean. Data points represent data from individual mice (young, wild-type, n=8; aged, wild-type, n=9; young, diabetic, n=7). Significant differences were determine using one-way analysis of variance and Dunnett's multiple comparisons test (*p<0.05).

FIG. 8A through FIG. 8D depict a high-frequency ultrasound imaging system. FIG. 8A depicts a 58-MHz single-element focused immersion transducer, and Pure View™ H pulser-receiver designed and manufactured by Imaginant, Inc. FIG. 8B depicts transducer characteristics. FIG. 8C depicts the first reflected ultrasound pulse measured using a steel reflector at the focus. FIG. 8D depicts the reference power spectrum measured using a steel reflector at the focus.

FIG. 9 depicts the anal sphincter complex anatomy.

FIG. 10 depicts an exemplary embodiment of a quantitative ultrasound imaging system.

FIG. 11 depicts an exemplary embodiment of a method of beam steering.

FIG. 12A through FIG. 12F depict an exemplary embodiment of a method of integrated backscatter coefficient estimation. FIG. 12A depicts a process of acquiring a backscattered echo in the time domain segmented by Hanning sub-windows. FIG. 12B depicts an exemplary time domain segment converted into a power spectrum of backscattered echo, S(f), in the frequency domain. FIG. 12C depicts an exemplary receive system's transfer function S_{ref}(f). FIG. 12D depicts exemplary attenuation corrections, A_{S}(f), A_{F}(f), and A_{M}(f), made for skin, fat, and muscle, respectively. FIG. 11E depicts an exemplary normalized power spectrum W(f). FIG. 12F depicts the computation of BCS(f) and IBC for each sub-window.

FIG. 13 depicts B-mode images of a linearly aligned suture fiber skeletal muscle phantom at insonification angles of 90°-85° in 1° increments.

FIG. 14A through FIG. 14B depict an anal sphincter complex phantom model for 3-D printing. FIG. 14A depicts a schematic of an anal sphincter complex for 3-D printing. FIG. 14B depicts a sagittal plane of an anal sphincter complex phantom.

FIG. 15 depicts an exemplary embodiment of a high-frequency ultrasound image acquisition setup of an anal sphincter complex ex vivo.

FIG. 16A through FIG. 16E depicts IBC parametric images and IBC as a function of insonation angle for rabbit puborectalis muscle imaged at 58-MHz ex-vivo. FIG. 16A depicts IBC parametric ROIs overlaid onto a B-mode image of rabbit puborectalis muscle at an insonation angle of 90°. FIG. 16B depicts IBC parametric ROIs overlaid onto a B-mode image of rabbit puborectalis muscle at an insonation angle of 85°. FIG. 16C depicts IBC parametric ROIs overlaid onto a B-mode image of rabbit puborectalis muscle at an insonation angle of 80°. FIG. 16D depicts the average IBC in an ROI as a function of insonation angle for rabbit puborectalis muscle. FIG. 16E depicts a dissection micrograph identifying the anatomy near the rabbit anal canal including the mucosa, fat tissue, and region of the puborectalis muscle.

FIG. 17 depicts an exemplary method for using ultrasound to characterize tissue structure.

FIG. 18 depicts an exemplary method for correlating ultrasound imaging data to measured tissue properties.

FIG. 19A through FIG. 19D depict B-mode images of porcine median nerve imaged ex-vivo using the Clarius L20 linear array transducer with a transmit frequency of 14 MHz. FIG. 19A depicts a B-mode image of porcine median nerve at an insonation angle of 90°. FIG. 19B depicts a B-mode image of porcine median nerve at an insonation angle of 85°. FIG. 19C depicts a B-mode image of porcine median nerve at an insonation angle of 80°. FIG. 19D depicts a B-mode image of porcine median nerve at an insonation angle of 75°.

FIG. 20A through FIG. 20D depict B-mode images showing a sagittal cross-section of flexor tendons at rest and under flexion in the human hand using the Clarius L20 clinical linear array transducer. The transmit frequency was 14 MHz. FIG. 20A depicts a B-mode image processed by the Clarius system with the hand at rest. FIG. 20B depicts a B-mode image computed from a new radiofrequency (RF) data exported from the Clarius. FIG. 20C depicts a B-mode image processed by the Clarius system with the hand under finger flexion. FIG. 20D depicts a B-mode image computed from raw radiofrequency (RF) data with the hand under finger flexion.

FIG. 21A through FIG. 21E depicts IBC parametric images and IBC as a function of insonation angle for rabbit external anal sphincter imaged at 58-MHz ex vivo. FIG. 21A depicts IBC parametric ROIs overlaid onto a B-mode image of rabbit external anal sphincter at an insonation angle of 90°. FIG. 21B depicts IBC parametric ROIs overlaid onto a B-mode image of rabbit external anal sphincter at an insonation angle of 85°. FIG. 21C depicts IBC parametric ROIs overlaid onto a B-mode image of rabbit external anal sphincter at an insonation angle of 80°. FIG. 21D depicts the average IBC in an ROI as a function of insonation angle for rabbit external anal sphincter. FIG. 21E depicts a dissection micrograph identifying the layers of the rabbit anal canal including the mucosa, serosa, and external anal sphincter.

FIG. 22A through FIG. 22E depict IBC parametric images and IBC as a function of insonation angle for porcine median nerve imaged at 58-MHz ex vivo. FIG. 22A depicts an IBC parametric ROI overlaid onto a B-mode image of porcine median nerve at an insonation angle of 90°. FIG. 22B depicts an IBC parametric ROI overlaid onto a B-mode image of porcine median nerve at an insonation angle of 85°. FIG. 22C depicts an IBC parametric ROI overlaid onto a B-mode image of porcine median nerve at an insonation angle of 80°. FIG. 22D depicts the average IBC in an ROI as a function of insonation angle for porcine median nerve. FIG. 22E depicts a dissection micrograph showing aligned tissue structure at the surface of porcine median nerve.

FIG. 23A through FIG. 23E depicts IBC parametric images and IBC as a function of insonation angle for porcine transverse carpal ligament imaged at 58-MHz ex vivo. FIG. 23A depicts an IBC parametric ROI overlaid onto a B-mode image of porcine transverse carpal ligament at an insonation angle of 90°. FIG. 23B depicts an IBC parametric ROI overlaid onto a B-mode image of porcine transverse carpal ligament at an insonation angle of 85°. FIG. 23C depicts an IBC parametric ROI overlaid onto a B-mode image of porcine transverse carpal ligament at an insonation angle of 80°. FIG. 23D depicts the average IBC in an ROI as a function of insonation angle for porcine transverse carpal ligament. FIG. 23E depicts a dissection micrograph showing aligned tissue structure at the surface of porcine transverse carpal ligament.

FIG. 24 depicts an exemplary computing environment in accordance with some of the embodiments.

FIG. 25 depicts a graph showing ultrasound metrics and tendon material properties. Tensile mechanical testing protocols were used to measure stress vs strain in a porcine tendon sample (black curve). Ultrasound backscatter intensity was measured simultaneously in the tendon during mechanical testing (red curve). Metrics associated with ultrasound backscatter intensity, measured noninvasively and nondestructively, can serve as surrogate predictors of tissue mechanical properties such as transition strain (red star), yield strain (green triangle), and Young's modulus (slope of linear region of ultrasound intensity versus strain).

### DETAILED DESCRIPTION

The following discussion omits or only briefly describes conventional features of methods, systems, and devices for medical imaging and characterization of tissue structure that are apparent to those skilled in the art. Those of ordinary skill may thus recognize that other elements may be desirable and/or necessary to implement the devices, systems, and methods described herein. It is noted that various examples are described in detail with reference to the drawings. Reference to these various examples does not limit the scope of the claims attached hereto. Additionally, any examples set forth in this specification are intended to be non-limiting and merely set forth some of the many possible implementations for the appended claims. Further, particular features described herein can be used in combination with other described features in each of the various possible combinations and permutations. As such, it is understood that the detailed description is exemplary and explanatory only and is not restrictive of the broad inventive concepts upon which the examples disclosed herein are based.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in the specification and the appended claims, the singular forms “a,” “an” and “the” include plural referents unless otherwise specified. The terms “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “operatively” or “operably connected” indicates such an attachment, coupling or connection that allows the pertinent structures to operate as intended by virtue of that relationship.

Reference throughout the specification to “exemplary”, “one example”, “an example” or “some examples” means that a particular feature, structure, or characteristic described in connection with at least one example of the subject matter disclosed. Thus, the appearance of the phrases “in one example”, “in an example” or “in some examples” in various places throughout the specification is not necessarily referring to the same example. Further, the particular features, structures or characteristics of “one example”, “an example” or “some examples” may be combined in any suitable manner with each other to form additional examples of such combinations. It is intended that examples of the disclosed subject matter cover modifications and variations thereof. Terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Moreover, throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range. As used herein, the term “about” in reference to a measurable value, such as an amount, a temporal duration, and the like, is meant to encompass variations of plus or minus 20%, plus or minus 10%, plus or minus 5%, plus or minus 1%, and plus or minus 0.1% of the specified value, as such variations are appropriate.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment or interest, while “distal” refers to a position that is situated away from the center of the body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The present invention discloses novel systems, methods, and devices for medical, imaging, analyzing tissue structures, and/or monitoring patient health. Although exemplary devices, methods, and systems for medical imaging, analyzing tissue structures, and/or monitoring patient health are disclosed, it should be appreciated that aspects of the devices, systems, and methods disclosed herein may be operated in conjunction with other systems or devices, including any medical imaging systems or devices, drug administration systems or devices, surgical systems or devices, surgical tools and/or instruments, tissue repair therapies, tendon repair therapies, and/or patient health monitoring devices.

In some aspects, the invention relates to an ultrasound-based method for rapid, non-invasive characterization of tissue microstructure. The method detects microstructural changes in tissues that may be associated with disease or aging. Certain tissues may have unique organizational structures that may be detected. As one example, in tendon, collagen fiber alignment is a key structural property that contributes to function. A single tendon is comprised of a hierarchy of parallel cylindrical subunits of decreasing diameter, namely fascicle, fiber, fibril, and collagen molecule. This hierarchical alignment can be altered in response to diabetes, aging, and injury. As a second example, a similar hierarchical microstructure occurs in neural tissue where a single nerve is comprised of multiple fascicles which are each comprised of multiple axons. This microstructural alignment of nerve can be affected by injury and disease. There are also other tissues that possess microstructural alignment and/or tissue hierarchy.

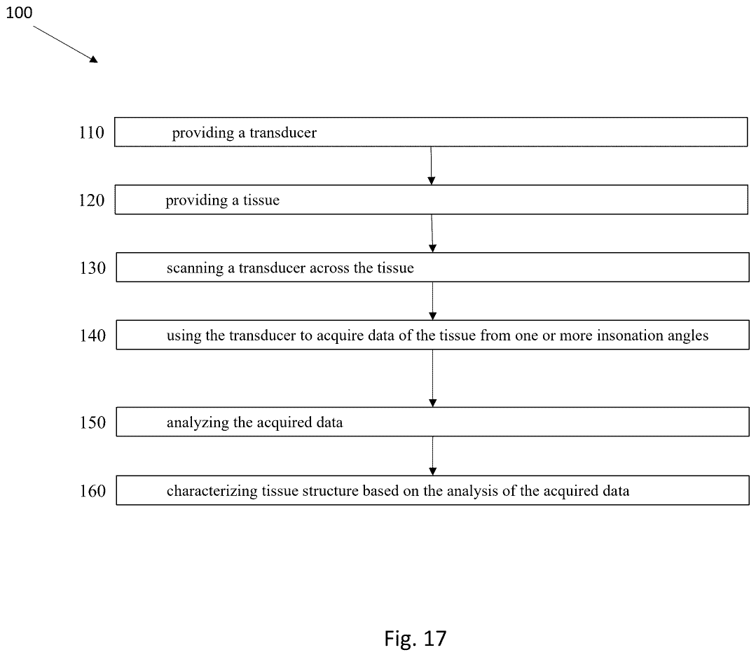

In some aspects, the invention relates to a method for point-of-care ultrasound imaging to quantitatively characterize tissue microstructure. The method may be a valuable clinical tool for diagnosis of microstructural changes in tissue, and for longitudinal monitoring of tissue healing to guide rehabilitation. In some aspects, the invention relates to a method of using ultrasound images of a tissue acquired at one or more angles to characterize tissue structure. Referring now to FIG. 17, shown is an exemplary method 100 for using ultrasound to characterize tissue structure. In some embodiments method 100 comprises the steps of 110 providing a transducer; 120 providing a tissue; 130 scanning a transducer across the tissue; 140 using the transducer to acquire data of the tissue from one or more insonation angles; 150 analyzing the acquired data; and 160 characterizing tissue structure based on the analysis of the acquired data.

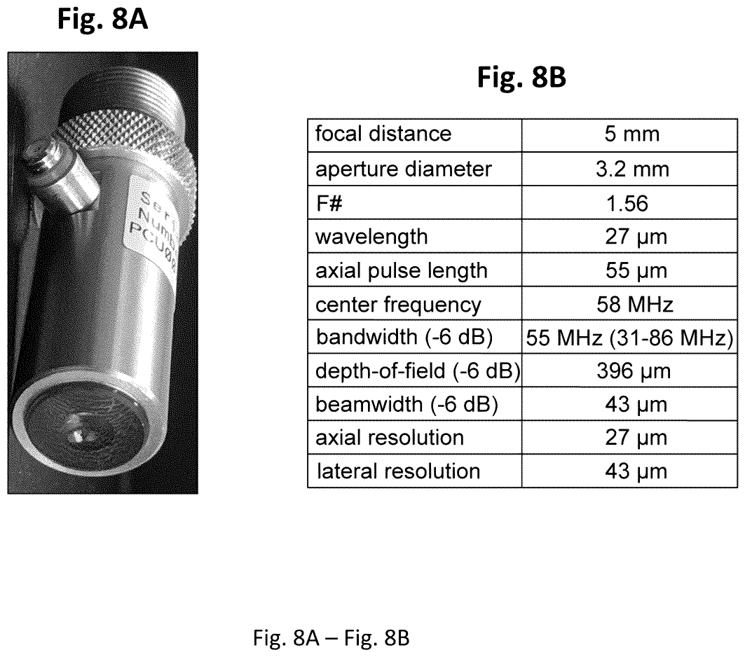

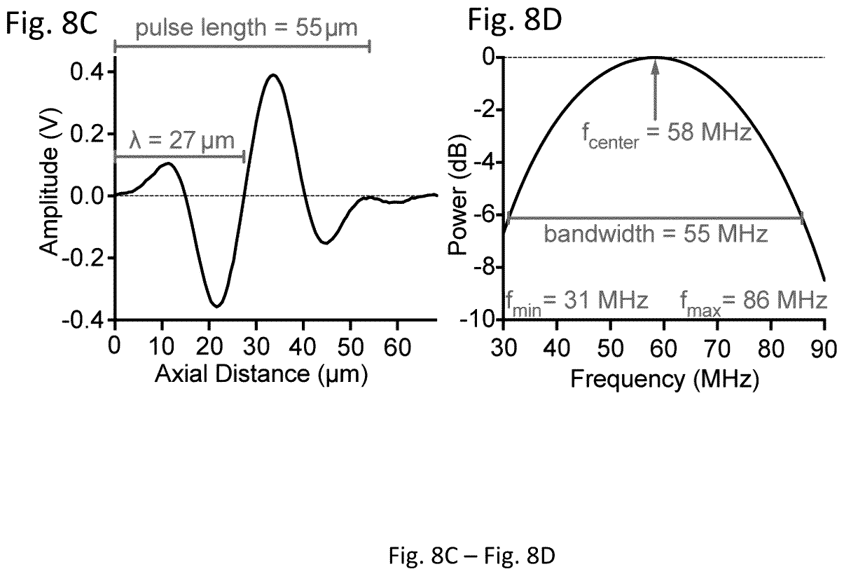

In some embodiments of method 100 any transducer is provided. The transducer may be a single-element transducer, for example, a single-element high frequency transducer, a 58-MHz single-element transducer, a focused immersion transducer, or a 58-MHz single-element focused immersion transducer. The transducer may be a 1.5D array transducer, a 2D array transducer, a ring array transducer, or an array transducer with any array geometry. In some embodiments, a transducer is used with a Vantage Research Ultrasound System. In some embodiments, the transducer is a linear-array ultrasound device used with a transmit frequency of 14 MHz. In some embodiments, the transducer is a Clarius L20 ultra-high frequency, linear-array ultrasound device. As in FIG. 8, the transducer may be a 58-MHz, single-element, focused immersion transducer or any similar transducer. The transducer may have any aperture diameter. For example, the transducer may have an aperture diameter of 3.2 mm as in FIG. 8. The transducer may have any F #. For example, as in FIG. 8, the transducer may have an F # of 1.56. In some embodiments of the method 100 the provided transducer is an array transducer, for example an 8-MHz linear array transducer or any low frequency linear array transducer. A low frequency transducer may have greater penetration depth when imaging a tissue in a subject. In some embodiments, the transducer may be provided along with a pulser-receiver. In some embodiments, the pulser receiver may trigger the firing of the transducer at any pulse repetition frequency, for example 200 Hz. In some embodiments, an oscilloscope may also be provided. The pulser receiver may amplify impulse responses with any receive gain, for example 17 dB, for display on an oscilloscope with any sampling frequency, for example a sampling frequency of 2.5 GHz. Acquired impulse responses may be an average of any number of received backscattered echoes, for example 10 received echoes, to optionally improve the signal to noise ratio.

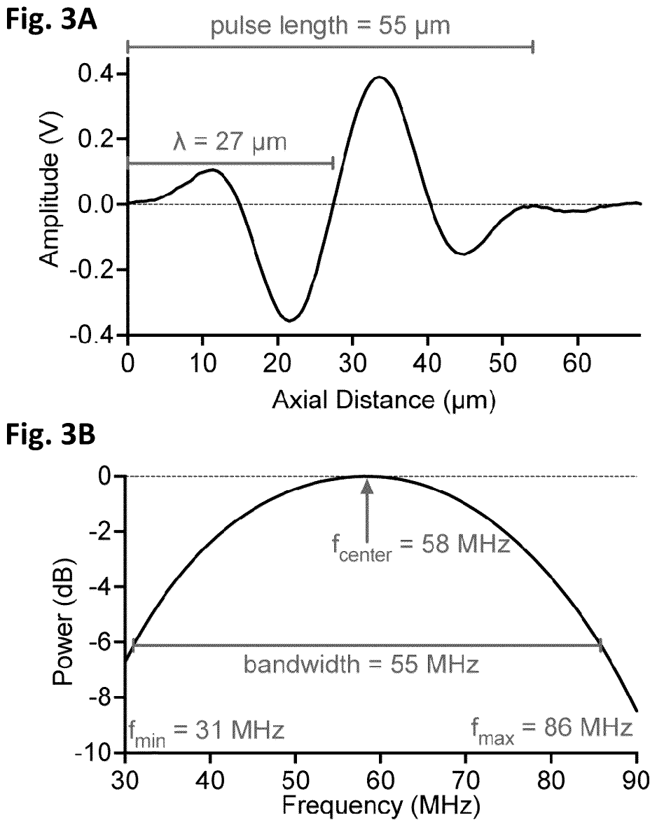

The transducer may have any focal distance. For example, as in FIG. 3A, the focal distance may be 5 mm. The transducer may emit a pulse with any wavelength and pulse length. For example, as in FIG. 3A, the wavelength may be 27 μm and the pulse length may be 55 μm. The transducer may have any depth-of-field. For example, the depth-of-field (−6 dB) may be 396 μm. The transducer may have any reference power spectrum. For example, as in FIG. 3B, the reference power spectrum may have a center frequency of 58 MHz and a −6 dB bandwidth of 55 MHz. The transducer may have any beamwidth. For example, the −6 dB beamwidth may be 43 μm.

In some embodiments of the method 100 any tissue is provided. The tissue may be a human tissue, an animal tissue, or a synthetically engineered tissue. The tissue may be a tissue having generally aligned collagen, for example tendon, ligament, cartilage, skeletal muscle, smooth muscle, cornea or nerve. The tissue may be any ligament or tendon, for example Achilles tendon, any flexor tendons, any flexor digitorum longus tendons, or any superficial tendons. Superficial tendons may be under skin and fat of a subject but may be above bone. The provided tissue may be a digital flexor tendon. The provided tissue may be a tendon of subject and the tendon may be at rest. The provided tissue may be a tendon of subject during tendon flexion. The subject may be instructed to flex or rest the tendon. The tissue may be a transverse carpal ligament. The tissue may be a median nerve or any nerve. The tissue may be the anal sphincter complex or any tissue that makes up the anal sphincter complex. The tissue may be a pediatric anal sphincter complex. The tissue may be the puborectalis muscles or any muscle that makes up the puborectalis muscles. The tissue may be from a healthy or diseased subject, for example the tissue may be from a subject who has a congenital anomaly, a congenital hindgut anomaly, diabetes, an autoimmune disease, a ruptured tendon, a scarred tendon, neuropathy, neural damage, or a ruptured muscle. The provided tissue may be part of a subject or the provided tissue may be surgically removed from a subject. The provided tissue may be part of a subject while the subject is at rest or while to subject is performing any functional movement. The provided tissue may be strained, may be under stress, or may be under any mechanical perturbation. The tissue may be of any region of the body.

In some embodiments of the method 100, the transducer is scanned across the tissue. The tissue may be positioned in any manner before the transducer is scanned across the tissue. In some embodiments, the tissue is positioned on a sample holder optionally after being surgically removed from a subject. In some embodiments, the sample holder and positioned tissue are placed in a liquid bath. In some embodiments, the liquid in the liquid bath is phosphate-buffered saline, water, any buffer solution, or any saline solution. In some embodiments the liquid bath is at room temperature or any temperature. In some embodiments, hair is removed from a tissue before the transducer is scanned across the tissue. In some embodiments, the sample holder has an acoustic window to prevent reverberations. In some embodiments, the angle of the tissue surface relative to the transducer is adjustable such that data can be acquired at multiple insonation angles.

In some embodiments, the transducer is scanned across a subject to acquire data of a tissue that has not been surgically removed. The transducer may be scanned across a subject in any manner such that an ultrasound field is scanned across a tissue. For example, the subject may remain still as the ultrasound field is scanned across a tissue of interest of the subject. The subject may lay flat. The tissue may be a superficial tissue or the tissue may be at some depth below the surface of the subject. For example, the tissue may be below the skin of a subject, below the skin and fat of a subject, and/or above the bone of a subject. The subject may be secured while the transducer is scanned across the tissue. For example, a subject's arms, legs, hands, feet, neck, torso, fingers, toes, or head may be secured such that the tissue of interest remains stationary as the transducer is scanned across the tissue. The subject may position their arms, legs, hands, feet neck, torso, fingers, toes or head in a liquid bath similar to methods described for surgically removed tissues. In some embodiments, hair is removed from the subject's skin above the target tissue to aid in transducer data acquisition. In some embodiments, a subject may be administered an anesthetic to aid the subject in remaining still. In some embodiments, the transducer is scanned across a tissue while the subject is undergoing a surgical procedure. For example, the transducer may be scanned across a ligament or tendon before, during, and after the ligament or tendon is being surgically repaired. In some embodiments, the subject may move the tissue in a specific manner as the transducer is scanned across the image. For example, the tendon or muscle of a subject may be undergoing loading or unloading cycles as the transducer is scanned across the tissue. For example, the tendon of a subject may be flexed by the subject. In some embodiments, the subject may be performing any functional movement. In some embodiments, the subject may be scanned over time to monitor healing progress and guide rehabilitation therapies.

In some embodiments, the transducer may be scanned across a muscle, ligament or tendon of a subject at various levels of loading. For example, the subject may flex the muscle, ligament, or tendon to various degrees that relate to the level of loading and the transducer may be scanned across the muscle, ligament, or tendon while one or more levels of flexion occurs. In some embodiments, the level of loading relates to the degree of bending or straightening of a joint. In some embodiments, the level of loading relates to the load borne by the muscle, tendon, or ligament. For example the subject may exert any amount of force by way of the muscle, tendon, or ligament that relates to a certain level of loading.

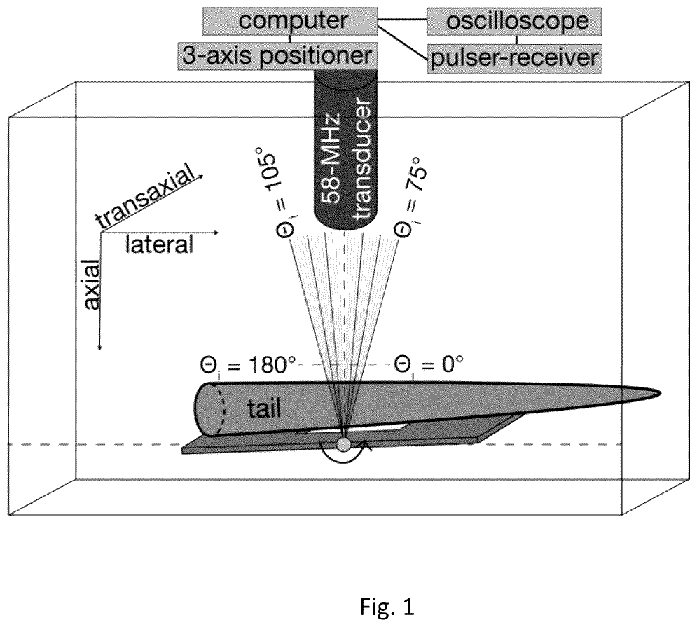

In some embodiments, the ultrasound field may be scanned across the tissue such that the transducer samples any number of insonation angles relative to the tissue. Any method known in the art for scanning of an imaging system across a subject may be used including any methods for electronic scanning of an imaging system. In some embodiments the transducer may be scanned across the tissue manually. For example, the transducer may be handled by an operator and scanned across a tissue at different angles relative to the surface of the tissue. The insonation angle may be defined as the angle between the incident ultrasound pulse and the tissue surface or skin surface. In some embodiments, the transducer is scanned across the tissue using a goniometer and/or a 3axis positioner. In some embodiments, a software may be used to position the transducer using the goniometer and 3-axis positioner. For example, the transducer may sample any number of angles relative to tissue surface in an automated fashion while scanning across the tissue. In an exemplary embodiment, the transducer may sample a range of insonation angles in which the midpoint of the range is 90°. In some embodiments, as in FIG. 1, the transducer samples insonation angles relative to the tissue surface ranging from 75° to 105°. In some embodiments, the transducer samples insonation angles relative to the tissue surface ranging from 73° to 109°. In some embodiments, the transducer acquires data at any number of insonation angles within a range. For example, data may be acquired at every 1° change in insonation angle. In some embodiments, the transducer samples an insonation angle of or of approximately 90° in addition to any number of other insonation angles. In some embodiments, data is acquired only at an insonation angle of or of approximately 90° and metrics acquired via analysis of this data are used as a metric of maximum backscatter strength.

In some embodiments, backscattered echoes are acquired by the transducer and pulser receiver at each sampled insonation angle. In some embodiments, backscattered echoes are acquired by electronic scanning of array transducers. In some embodiments, a Vantage 64LE system is used. For backscattered echo acquisition, the pulser-receiver may trigger the firing of the transducer at any pulse repetition frequency, for example 200 Hz. Backscattered echoes may be amplified with any receive gain, for example 50 dB, for display on an oscilloscope with any sampling frequency, for example 2.5 GHz. In some embodiments, A-lines are generated by averaging acquired backscattered echoes to improve the signal to noise ratio. For example, A-lines may be an average of 10 backscattered echoes. A-lines may be acquired in any step size. A-lines may be acquired at a step-size that is greater than the transducer beamwidth to ensure independence between neighboring A-lines. For example, A-lines may be acquired in 60 μm steps when the transducer beamwidth is 43 μm. In some embodiments, B-mode images may be generated. B-mode images may display acoustic intensity to provide sagittal cross-sectional images of a provided tissue. Independence between scatterers in neighboring planes may be confirmed. For example, lateral scans of two planes separated transaxially by 2 step sizes may be performed to ensure scatterers from one resolution volume do not interfere with scattering from a resolution cell volume in a neighboring plane.

In some embodiments, the integrated backscatter coefficient (IBC) is estimated for each sampled insonation angle. Any method for estimating IBC may be used. In some embodiments, each backscattered echo is segmented into sub-windows of any axial and lateral dimensions. For example, the sub-windows may be 55 μm axially by 60 μm laterally. In some embodiments, a Hanning window is used to extract echo segments in each sub-window in the time domain. Hanning window extraction may be used to reduce ringing in power spectra. In some embodiments, Hanning windows may be overlapped by any percentage, for example 50%, to compensate for signal suppression near window edges. Edge-detection algorithms may be designed to identify the surface of provided tissue using filtering or thresholding methods for example two-dimensional Gaussian lowpass filtering, median filtering, and/or intensity thresholding.

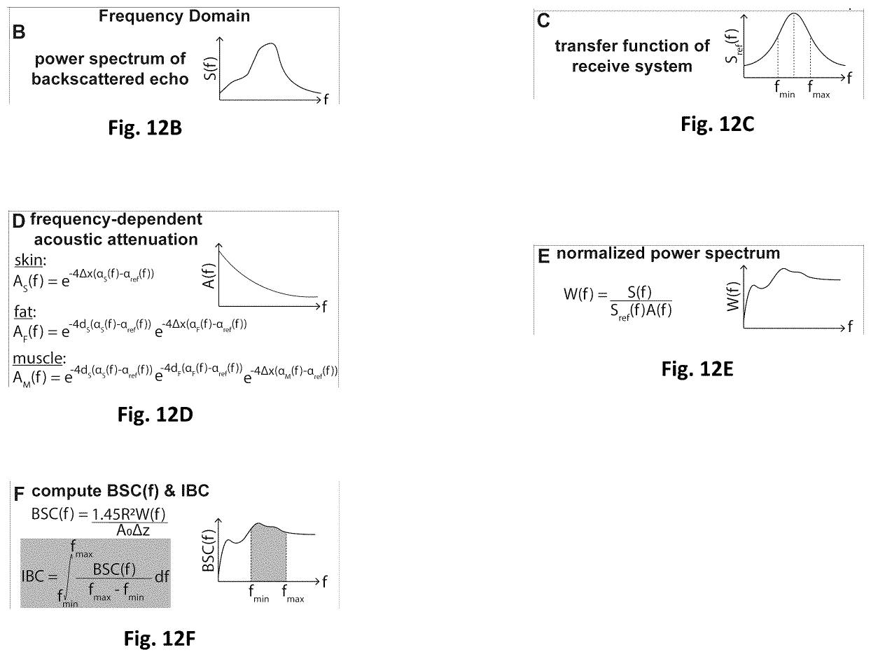

In some embodiments, the IBC is estimated as in FIG. 12. For example, the power spectrum of each backscattered echo segment, S(f), is computed and then divided by the transfer function of the receive system at each axial depth, S_{ref}(f), to account for the frequency response of the receive system. In some embodiments, a correction may be made to compensate for acoustic attenuation in tissue as a function of depth and frequency. For example, equations for normalized power can be written as follows for liver, W_{L}(f), skin, W_{S}(f), and tendon, W_{T}(f):

$${W}_{L} ( f ) = \frac{S ( f )}{{S}_{r e f} ( f )} {e}^{4 Δ {x}_{L} ( {α}_{L} - {α}_{r e f} )} \qquad ( 1 )$$

$${W}_{S} ( f ) = \frac{S ( f )}{{S}_{r e f} ( f )} {e}^{4 Δ {x}_{S} ( {α}_{S} - {α}_{r e f} )} \qquad ( 2 )$$

$${W}_{T} ( f ) = \frac{S ( f )}{{S}_{r e f} ( f )} {e}^{4 {d}_{S} ( {α}_{S} - {α}_{r e f} )} {e}^{4 Δ {x}_{T} ( {α}_{T} - {α}_{r e f} )} \qquad ( 3 )$$

where Δx_{L}, Δx_{S}, and Δx_{T} are propagation depths into liver, skin, and tendon, respectively. The skin thickness is denoted d_{S}. Attenuation coefficients of liver, skin, tendon, and reference medium are denoted α_{L}, α_{S}, α_{T}, and α_{ref}, respectively. Frequency-dependent attenuation coefficients may be expressed as ∝_{L} (f)=0.4f (dB cm⁻¹), ∝_{S} (f)=0.264f^{1.69} (dB cm⁻¹), ∝_{T} (f)=2.42f+3.90 (dB cm⁻¹), and ∝_{ref} (f)=0.0022f² (dB cm⁻¹).

In some embodiments, the backscatter coefficient, BSC(f), and IBC are computed following spectra normalization. BSC(f) is the ratio of backscattered to incident intensity per unit volume. BSC(f) may be expressed as

$$B S C ( f ) = \frac{1.45 W ( f ) {R}^{2}}{{A}_{0} Δ z} \qquad ( 5 )$$

where A₀ is the transducer's aperture area, R is the distance between the transducer and the top of the region-of-interest (ROI), and Δz is the depth of the ROI. The IBC can be expressed as:

$$IBC = {\int }_{{f}_{min}}^{{f}_{max}} \frac{B S C ( f )}{{f}_{max} - {f}_{min}} d f \qquad ( 6 )$$

where fₘᵢₙ and fₘₐₓ are the minimum and maximum frequencies of the transducer bandwidth (−6 dB), respectively.

The BSC(f) and IBC may be calculated for each subwindow of a provided tissue region of interest. In some embodiments, a software may be used to estimate the IBC in a provided tissue sample at multiple insonation angles.

The transducer acquired data may be analyzed. In some embodiments, the IBC is estimated at any number of insonation angles, and the relationship between IBC and insonation angle is determined. In other words, the estimated IBC as a function of insonification angle for a provided tissue may be quantified. One or more representative regions of interest (ROI) of a provided tissue with any axial and lateral dimensions may be chosen for quantifying estimated IBC as a function of insonation angle. For example, the dimensions of a tissue ROI may be 1 mm laterally by 250 μm, 200 μm, or 55 μm axially. The ROI dimensions may be chosen or optimized based on the tissue type. For example, the ROI may be 1 mm laterally by 250 μm or about 1 mm laterally by about 250 μm axially for tendon or liver. The ROI may be 1 mm laterally by 200 μm or about 1 mm laterally by about 200 μm axially for skin. The ROI may be 1 mm laterally by 55 μm axially or about 1 mm laterally by about 55 μm for tendon fascicles. Any number of A-lines may be provided for analyses per ROI. The number of A-lines may be proportional to the width of an ROI. For example, 16 A-lines may be provided for an ROI width of 1 mm. The ROI may remain within the theoretical depth-of-field of the transducer as the transducer insonation angle is changed.

In some embodiments, the IBC is estimated at an insonation angle of or of approximately 90°, and this estimated IBC is used as a metric of maximum backscatter strength.

Analyzing transducer acquired data may include using any method to quantify a relationship between IBC and insonation angle and/or any method to quantify or estimate maximum backscatter strength across all insonation angles. For example, to quantify a relationship between IBC and insonation angle, the average IBC over an ROI of a tissue, IBC_{ROI}, may be quantified for each insonation angle sampled by the transducer. BSC(f) and IBC for any insonation angle may be calculated for each ROI subwindow. For each insonation angle, the IBC of each ROI subwindow may be averaged to compute the average IBC over the entire ROI also denoted as IBC_{ROI}. In some embodiments, any descriptive statistic for the IBC over the entire ROI is calculated as a substitute for or in addition to IBC_{ROI}, for example, median or mode of each ROI subwindow estimated IBC. In embodiments in which the provided tissue is a tendon, the IBC_{ROI} may be normalized for the number of fascicle edges within the ROI. In some embodiments, the IBC_{ROI} is normalized to the number of any relevant anatomical features in an ROI that may increase or decrease IBC in any tissue type. This may be repeated for any number of insonation angles. The IBC_{ROI} may be plotted against corresponding insonation angle for each sampled insonation angle.

Any metric may be calculated to represent the dependence of IBC_{ROI} on insonation angle or in other words the angular dependence of scattering. Metrics of maximum backscatter strength may also be calculated as part of an analysis of transducer acquired data. In instances in which IBC_{ROI} is dependent on insonation angle a Gaussian function may be fitted to curves of IBC_{ROI} versus insonation angle. In many tissues especially tissues with aligned collagen or any other aligned microstructures, the maximum IBC_{ROI}, and in turn the peak of the fitted Gaussian function, occurs at or around 90°. In some embodiments, the maximum IBC_{ROI} (i.e., maximum backscatter strength) is simply calculated as the IBC_{ROI} that occurs at or around an insonation angle of 90°. In some embodiments, Gaussian functions are fitted to curves of IBC_{ROI} Versus insonation angle for multiple planes and then averaged to generate one Gaussian function. Planes may be separated by any distance, for example 120 μm. Gaussian functions may be fitted using any method and optionally with the aid of a software.

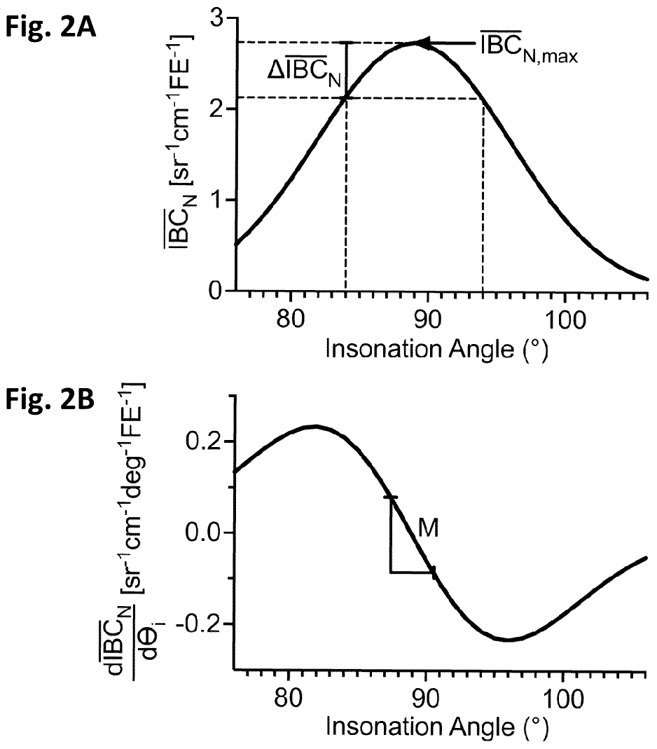

Any number of metrics may be calculated from the Gaussian fit of IBC_{ROI} versus insonification angle. The value of the function of the Gaussian fit of IBC_{ROI} Versus insonation angle is denoted as I̅B̅C̅_{N}. Any number of metrics may be calculated for scattering strength. For example, as in FIG. 2A, the maximum value of the Gaussian fit, denoted as I̅B̅C̅_{N,max}, may be used as a metric of scattering strength. Other metrics of scattering strength are contemplated herein including the maximum value of IBC_{ROI} across all sampled insonation values, the value of IBC_{ROI} at an insonation angle of 90° otherwise known as the normal insonation angle. Any number of metrics for the angular dependence of scattering may be calculated. For example, the change of I̅B̅C̅_{N} within 10° of I̅B̅C̅_{N}, max, denoted as ΔI̅B̅C̅_{N}, may be used as a metric of the angular dependence of the scattering strength. The change of I̅B̅C̅_{N} within any number of degrees of I̅B̅C̅_{N,max}, may also be used as a metric of the angular dependence of scattering strength. The derivative of I̅B̅C̅_{N},

$$\frac{d {\overline{IBC}}_{N}}{d {θ}_{i}} ,$$

may be plotted as a function of insonification angle. The slope, M, of the linear region of

$$\frac{d {\overline{IBC}}_{N}}{d {θ}_{i}}$$

as a function of angle may be used as an additional metric of the angular dependence of the IBC near normal incidence. Other contemplated metrics of the angular dependence of the scattering strength include the standard deviation of the gaussian fit, the maximum of the derivative of the Gaussian fit,

$$\frac{d {\overline{IBC}}_{N}}{d {θ}_{i}} ,$$

or the absolute value of the minimum of the derivative of the Gaussian fit,

$$\frac{d {\overline{IBC}}_{N}}{d {θ}_{i}} ,$$

qualitative descriptions of the Gaussian fit.

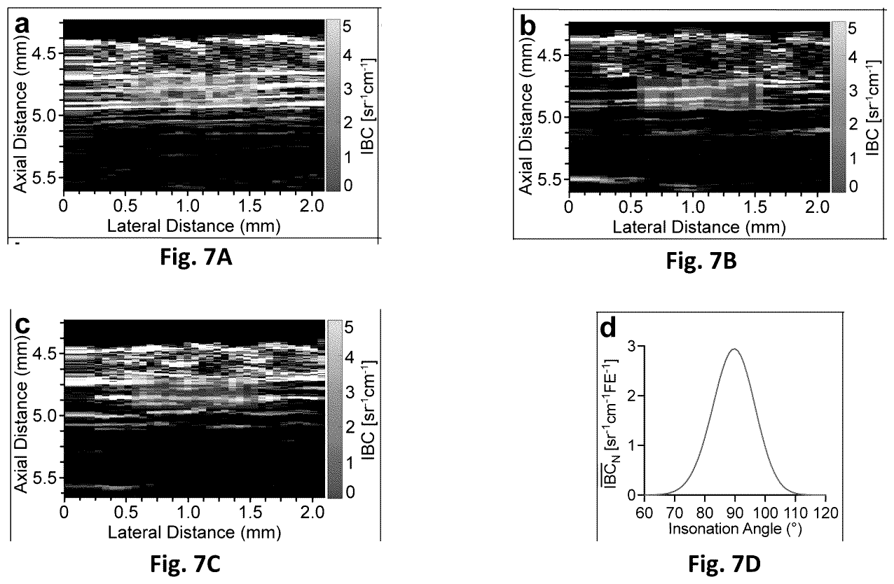

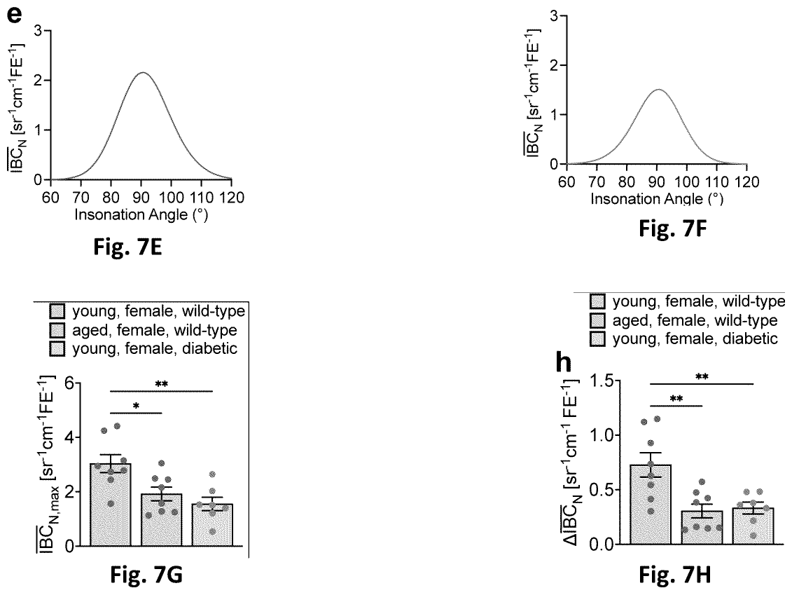

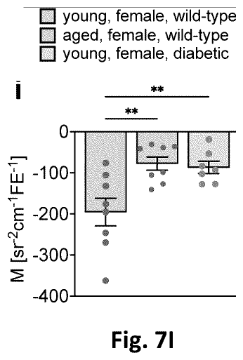

Analysis of the transducer acquired data may be used to characterize tissue structure. For example, any contemplated metrics of backscatter strength or angular dependence of scatter may be correlated with properties of tissue structure. For example, as in FIG. 7, backscatter strength and angular dependence of scatter may be indicative of tissue age or disease state. Greater backscatter strength or greater angular dependence of scatter may be indicative of a younger tissue or a healthier tissue. Lesser backscatter strength or lesser angular dependence of scatter may be indicative of a pathology or a disease state. Lesser backscatter strength or lesser angular dependence of scatter may be indicative of tissue over-use or a congenital malformation.

Any properties of tissue structure or microstructure may be characterized based on analysis of the transducer acquired data, for example metrics of backscatter strength or metrics of angular dependence of scatter. Contemplated properties of tissue structure or microstructure include cell alignment within tissues. For example, axonal alignment within a nerve, fibroblast alignment within ligaments, myocyte alignment within muscle, chondrocyte alignment within cartilage, cardiomyocyte alignment within heart, or cellular alignment within an artificially fabricated tissue. Contemplated properties of tissue structure or microstructure also include vascular alignment and vascular organization. Contemplated properties of tissue structure or microstructure also include extracellular matrix fiber organization including collagen fibers. Properties of extracellular matrix fiber organization may include, fiber alignment including the spread of extracellular matrix fiber directions, fiber length including average fiber persistence length, fiber density, fiber crosslinking, proportions of fiber types including collagen fiber subtypes, or fiber tension. Extracellular matrix fibers may include collagens, fibronectin, basement membrane, hyaluronic acid, or laminin. For example, increased fiber organization, alignment, length, or density may be correlated with increased angular dependence of scatter or increased backscatter strength.

Any tissue material properties may be characterized based on analysis of the transducer acquired data, for example metrics of backscatter strength or metrics of angular dependence of scatter. Contemplated tissue material properties include stiffness including young's modulus. Contemplated tissue material properties also include transition strain, transition stress, yield strain, yield stress, ultimate strain, ultimate stress, viscoelasticity, tensile strength, compressive strength, tensional load, and compressive load. For example, increased stiffness may be correlated with decreased angular dependence of scatter and decreased backscatter strength.

Any biological properties of tissue may be characterized based on analysis of the transducer acquired data, for example metrics of backscatter strength or metrics of angular dependence of scatter. Contemplated tissue properties may include inflammation, tissue age, tissue vascularization, tissue disease state, tissue fibrosis, tissue scarring, or tissue steatosis for example fatty muscle or fatty liver, tissue cancer state, tissue rupture including muscle or tendon rupture. For example, increased tissue age, inflammation, scarring, fibrosis, injury or steatosis may be correlated with a decrease in metrics of backscatter strength or decrease in metrics of angular dependence of scatter. In some examples a pathology or disease is associated with a decrease in metrics of backscatter strength or decrease in metrics of angular dependence of scatter. In some examples a tissue overuse or malformation is associated with a decrease in metrics of backscatter strength or decrease in metrics of angular dependence of scatter.

Any systemic states of a subject including disease states of a subject, subject age, or subject metabolic health may be characterized based on analysis of the transducer acquired data for example metrics of backscatter strength or metrics of angular dependence of scatter. Contemplated systemic states of a subject include any autoimmune disease, diabetes, obesity, congenital malformation for example congenital hindgut anomalies. For example, decreases in angular dependence may be correlated with the presence of an autoimmune disease, increased age, presence of diabetes, presence of a congenital malformation, or the presence of a metabolic disease.

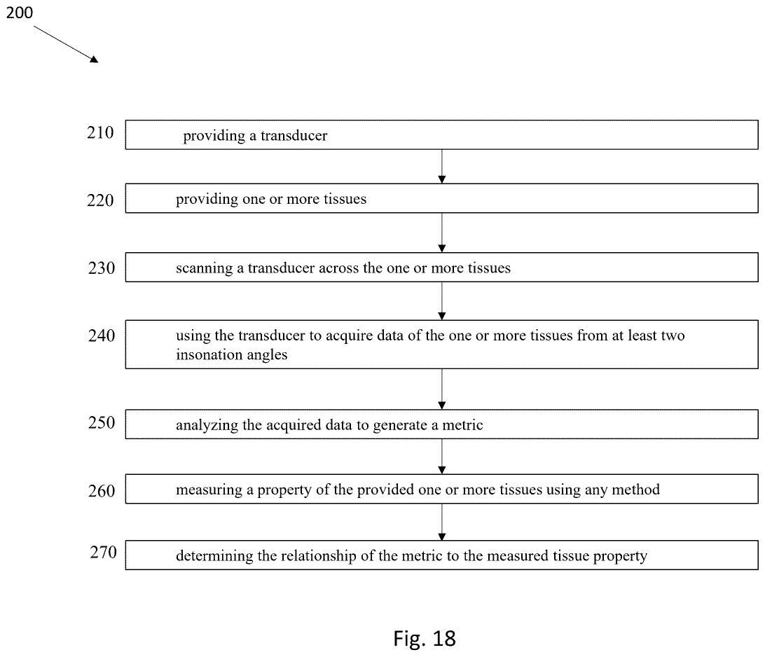

In some aspects, the invention relates to a method 200 for using ultrasound images of one or more tissues acquired at one or more angles to correlate an analysis of transducer acquired data, for example calculated metrics of backscatter strength or of angular dependence of scatter, to any measured tissue property. Referring now to FIG. 18, shown is an exemplary method 200. In some embodiments method 200 comprises the steps of 210 providing a transducer; 220 providing one or more tissue; 230 scanning a transducer across the one or more tissues; 240 using the transducer to acquire data of the one or more tissues from at least two insonation angles; 250 analyzing the acquired data to generate a metric; 260 measuring a property of the provided one or more tissues using any method; and 270 determining the relationship of the metric to the measured tissue property.

In some examples of the method 200 the measured tissue property is any property. For example, the measured tissue property may be any property of tissue structure or tissue microstructure, any qualitative tissue property, and tissue material property, or any cellular or biomolecular property of the tissue. For example, the tissue property may be measured using a tensile test, any histology approaches, any approaches to measure gene expression, or approaches to characterize the genome of organism from which the tissue is derived.

In some aspects, the invention relates to a point of care device. For example, the device may include a transducer, a pulser receiver, a mechanism to position the transducer, and a sample holder. The transducer may be positioned automatically optionally using a robotic system. The device may include robotics to move the transducer and for beam steering. The device may automatically position the transducer such that any number of insonation angles relative to a tissue of interest. Some embodiments of the device may include a software to allow the operator to set desired insonation angles. The software may also estimate IBC. The software may help the user optimize insonation angles, transducer properties, or IBC estimation based on different tissue types. The device may also include a processor that may execute the software. The device may also include a display to transmit information or communicate with the device operator or the subject.

In some aspects, the invention relates to a method of diagnosis of a subject. Diagnoses or risk assessments of any disease or adverse events may be based on analysis of the transducer acquired data, for example metrics of backscatter strength or metrics of angular dependence of scatter. Diagnosis of a disease or assessment of disease risk may involve a threshold value of a metric. Metabolic disease, diabetes, autoimmune disease, congenital anomalies, congenital hindgut anomalies may be diagnosed. Additionally, the risk of muscle or tendon injury including rupture and tears, the risk of fecal incontinence, the risk of developing a metabolic disease, or the risk of developing an autoimmune disease may be assessed. For example, increases in angular dependence or backscatter strength may be correlated with a decreased risk of muscle or tendon injury, decreased risk of fecal incontinence, or the development of any disease.

In some aspects, the invention relates to a method of guiding rehabilitation of a subject from an injury. For example, metrics of backscatter strength or metrics of angular dependence of scatter may be acquired longitudinally as a subject recovers from an injury to guide treatment decisions or any rehabilitation regimen. For example, the recovery of an injured muscle, tendon, or nerve of a subject may be monitored longitudinally during recovery. Metrics of backscatter strength or metrics of angular dependence of scatter may be used to characterize the recovery of the muscle, tendon, or nerve. For example, greater backscatter strength or angular dependence may be correlated with increased recovery and/or decreased risk of reinjury.

In some examples, analysis of transducer acquired data from a tendon or muscle of a subject at rest may be compared to transducer acquired data from the same tendon or muscle of the same subject undergoing tendon flexion, performing a functional movement, or loading a muscle. The change in any transducer acquired data, or any analysis of transducer acquired data including metrics of backscatter strength or metrics of angular dependence of scatter may be compared between the tissue at rest and the tissue undergoing loading, flexion, or during a functional movement. The change in metrics of backscatter strength or metrics of angular dependence of scatter compared between these two states may inform the disease state of a subject, disease state of a tissue, injury state of a tissue, the strength of a tissue, recovery of a tissue from injury, or the risk of injury of a tissue.

In some examples, transducer acquired data from a tendon or muscle of a subject at various levels of flexion or loading of tissues such as a tendon, ligament or muscle, may be analyzed. For example, analyzing data acquired at different levels of tissue flexion or loading may aid in guiding rehabilitation of the subject optionally from a tendon, ligament, or muscle injury. For example, the subject may flex or load a tendon, ligament, or muscle as data is acquired via transducer and optionally subsequently analyzed. The analysis may indicate to the subject or a professional such as a physician, therapist, or anyone aiding in the rehabilitation of the subject the level of flexion or loading that can be safely performed by the subject. For example, the analysis may indicate that the subject has reached a maximum level of flexion or loading that has no risk or minimal risk of injury or reinjury. In some embodiments, the maximum level of safe flexion or loading may change including increase or decrease over time or over any number of rehabilitation sessions.

In some embodiments, the level of tendon flexion of muscle loading relates to stress and/or strain of the tendon or muscle. In some embodiments, a subject may be prompted to flex a tendon or to load a muscle. For example, tendon flexion or muscle loading may include bending or straightening a joint to reach any degree of bending, moving any body part, exerting any force via any body part or muscle, holding any amount of weight, or gripping on object by any body part. The degree of joint bending, body part movement, force exertion, amount of weight being held, or grip exerted by the subject may relate to the level of tendon flexion or muscle loading.

For example, any metrics of backscatter strength or angular dependence of scatter contemplated herein may be plotted against the level of flexion or loading of the tendon or muscle, respectively. Similarly, any metrics of backscatter strength or angular dependence of scatter contemplated herein may be plotted against the stress or strain of a tendon or muscle. Stress or strain may be calculated from the level of flexion or loading of the tissue. Comparing metrics across levels of tissue flexion, loading, stress, and/or strain may be used to determine material properties of the tendon or muscle.

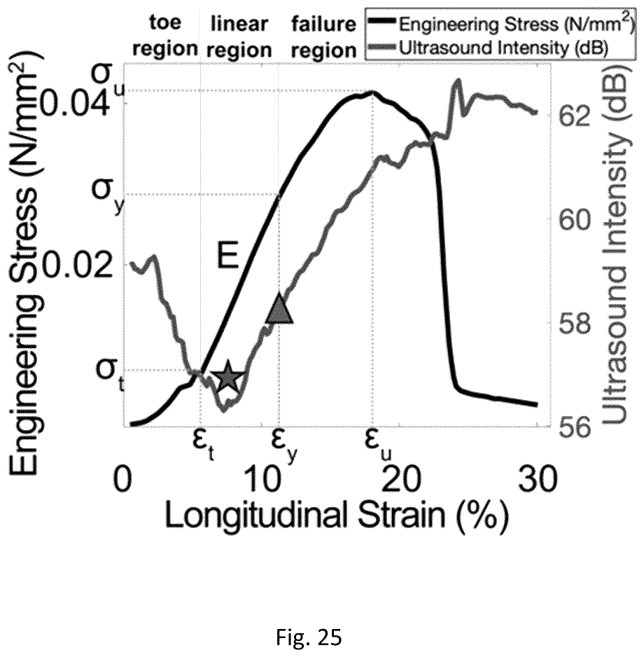

For example, a plot of a metric derived from analysis of transducer data against flexion, loading, stress, or strain of the tissue of the subject may reveal any relationship. In some embodiments, the plot may reveal different relationships in different regimes of flexion, loading, stress, or strain. In some embodiments, a transition point between any regime of flexion, loading, stress, or strain may indicate a level of maximum safe loading or safe flexion. Exemplary regimes of flexion, loading, stress, and/or strain, may include a toe region, linear region, and failure region. In some embodiments, the transition point relates to the beginning or end of a linear region of the plot. In some embodiments, the plot may comprise a toe region, linear region, and failure region, for example as in FIG. 25. In some embodiments, the transition point relates to the transition between the toe region and linear region or the linear region and failure region. In some embodiments, the transition point relates to the transition stress, transition strain, transition flexion, and/or transition load of the tissue. For example, the lowest stress, strain, flexion, or loading, of a linear region of the plot may relate to the transition stress, transition strain, transition flexion, or transition load. For example, the point of transition between a toe region and linear region of the plot may relate to the transition stress, transition strain, transition flexion, or transition load. In some embodiments, the transition point relates to the ultimate stress, ultimate strain, ultimate flexion, and/or ultimate load of the tissue. For example, the greatest stress, strain, flexion, or loading of a linear region of the plot may relate to the ultimate stress, ultimate strain, ultimate flexion, or ultimate load. For example, the point of transition between a linear region and failure region of the plot may relate to the ultimate stress, ultimate strain, ultimate flexion, or ultimate load. In some examples, the slope of a linear region of the plot relates to the Young's modulus of the tissue.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

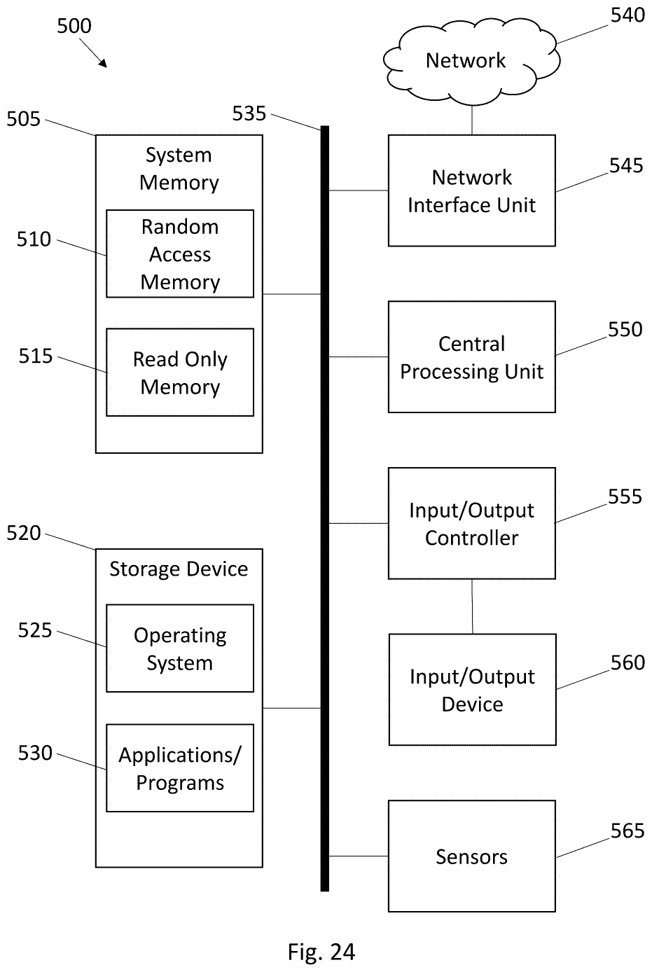

FIG. 24 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 24 depicts an illustrative computer architecture for a computer 500 for practicing the various embodiments of the invention. The computer architecture shown in FIG. 24 illustrates a conventional personal computer, including a central processing unit 550 (“CPU”), a system memory 505, including a random-access memory 510 (“RAM”) and a read-only memory (“ROM”) 515, and a system bus 535 that couples the system memory 505 to the CPU 550. A basic input/output system containing the basic routines that help to transfer information between elements within the computer, such as during startup, is stored in the ROM 515. The computer 500 further includes a storage device 520 for storing an operating system 525, application/program 530, and data.

The storage device 520 is connected to the CPU 550 through a storage controller (not shown) connected to the bus 535. The storage device 520 and its associated computer-readable media, provide non-volatile storage for the computer 500. Although the description of computer-readable media contained herein refers to a storage device, such as a hard disk or CD-ROM drive, it should be appreciated by those skilled in the art that computer-readable media can be any available media that can be accessed by the computer 500.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 500 may operate in a networked environment using logical connections to remote computers through a network 540, such as TCP/IP network such as the Internet or an intranet. The computer 500 may connect to the network 540 through a network interface unit 545 connected to the bus 535. It should be appreciated that the network interface unit 545 may also be utilized to connect to other types of networks and remote computer systems.

The computer 500 may also include an input/output controller 555 for receiving and processing input from a number of input/output devices 560, including a keyboard, a mouse, a touchscreen, a camera, a microphone, a controller, a joystick, or other type of input device. Similarly, the input/output controller 555 may provide output to a display screen, a printer, a speaker, or other type of output device. The computer 500 can connect to the input/output device 560 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 520 and RAM 510 of the computer 500, including an operating system 525 suitable for controlling the operation of a networked computer. The storage device 520 and RAM 510 may also store one or more applications/programs 530. In particular, the storage device 520 and RAM 510 may store an application/program 530 for providing a variety of functionalities to a user. For instance, the application/program 530 may comprise many types of programs such as a word processing application, a spreadsheet application, a desktop publishing application, a database application, a gaming application, internet browsing application, electronic mail application, messaging application, and the like. According to an embodiment of the present invention, the application/program 530 comprises a multiple functionality software application for providing word processing functionality, slide presentation functionality, spreadsheet functionality, database functionality and the like.

The computer 500 in some embodiments can include a variety of sensors 565 for monitoring the environment surrounding and the environment internal to the computer 500. These sensors 565 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

### Example 1: Quantitative Ultrasound for Characterizing Tendon Structure Using the Angular Dependance of Integrated Backscatter

A high-frequency, quantitative ultrasound spectral analysis technique to characterize collagen microstructure in tendon non-invasively and non-destructively was developed. The feasibility of employing the angular dependence of the integrated backscatter coefficient (IBC) to detect alterations in tendon microstructure in the presence of diabetes and with aging was investigated in murine tendon. Backscattered echoes were obtained from tail tendon, overlying tail skin, and liver of female mice at 31 insonation angles using a single-element, 58-MHz transducer. Parametric images of IBC values, and the average IBC for a region-of-interest were computed at each angle. IBC measurements were dependent on angle in tendon with aligned collagen fibers and were independent of angle in skin and liver. Quantitative ultrasound metrics characterizing the IBC as a function of angle included (1) I̅B̅C̅_{N,max}, the maximum IBC, (2) ΔI̅B̅C̅_{N}, the change in the IBC within ten degrees of its maximum, and (3) M, the linear rate of change of the derivative of the IBC as a function of insonation angle. All three quantitative ultrasound metrics were significantly lower in diabetic tendon and aged tendon compared to wild-type tendon, indicating that scattering strength was reduced, and tendon disorganization was increased in tail tendons of diabetic mice and aged mice compared to wild-type mice. This work demonstrated the utility of an ultrasound technique employing metrics associated with the IBC to characterize tendon microstructure non-invasively.

A high-frequency, quantitative ultrasound system and spectral analysis technique to characterize collagen microstructure in healthy, diabetic, and aged murine tendon was developed. It was hypothesized that the IBC exhibits angular dependence in tendon with aligned structure, and angular independence in tissues with heterogeneous structure. Furthermore, it was hypothesized that quantitative metrics derived from the magnitude and angular dependence of the IBC can effectively discern differences in tendon microstructure in diabetic and aged mice compared to young, healthy mice. IBC parametric images were computed from backscattered echoes acquired over a range of insonation angles. Measurements with young, wild-type mice were first completed to demonstrate differences in the angular dependence of the IBC in tendon compared to heterogeneous skin and liver tissues. Angular-dependent scattering characteristics of tendon were also demonstrated at the intratendon and intrafascicular levels. Three metrics derived from the magnitude and angular dependence of the IBC were then developed and employed to quantify differences in tendon microstructure in the presence of diabetes and with aging. This quantitative ultrasound tissue characterization technique may be used in a point-of-care device for longitudinal monitoring of tendon injury and repair to guide rehabilitative approaches for tendon healing in healthy, diabetic, and aged patients.

The methods are now described.

### Murine Tissue Sample Preparation

Genetically-diabetic mice (BKS.Cg-Dock7^{m}+/+Lepr^{db}/J; 10-16 weeks; female) and wild-type mice (C57BL/6J; 10-16-weeks; female) were obtained from The Jackson Laboratory (Bar Harbor, ME, USA). Aged, wild-type mice (C57BL/6J; 20-25 months; female) were acquired from the National Institute on Aging of the National Institutes of Health (Charles Rivers Laboratories; Wilmington, MA). All animal protocols were in accordance with policies of the University Committee on Animal Resources at the University of Rochester. Mice were sacrificed and hair was removed from the tail skin using a depilatory cream. Tails were removed and livers were dissected from the abdominal cavity. For ultrasound imaging, tissues were positioned on a sample holder in a degassed phosphate-buffered saline (PBS) bath at room temperature. The sample holder had an acoustic window to prevent reverberations, and the angle of the tissue surface relative to the transducer was adjustable to enable acquisition of backscattered echoes as a function of insonation angle.

### Transducer Characterization

A 58-MHz, single-element, focused immersion transducer and pulser-receiver were designed and manufactured by Imaginant, Inc. (now Byk-Gardner, Pittsford, NY) (FIG. 8). The transducer had a 3.2-mm aperture diameter and an F # of 1.56 (FIG. 8B). The first reflected ultrasound pulse and reference power spectrum were measured using a steel reflector at the focus (FIG. 8C-8D). A goniometer (Newport Corporation, Irvine, CA), three-axis-positioner (Velmex, Inc., Bloomfield, NY), and custom Matlab® program (Mathworks, Inc., Natick, MA) were used to align and position the transducer normal to a steel reflector located at the 5-mm focal distance. The pulser-receiver triggered the firing of the transducer at a pulse repetition frequency of 200 Hz and amplified impulse responses with a receive gain of 17 dB for display on the oscilloscope (Waverunner 62 Xi-A, LeCroy Corp., Chestnut Ridge, NY) with a sampling frequency of 2.5 GHz. Each acquired impulse response was an average of 10 received echoes to improve the signal to noise ratio (SNR). The receive system impulse response was acquired at the focal distance, and wavelength and pulse duration were measured. Impulse responses acquired axially were used to estimate depth-of-field. Impulse responses were acquired within the depth-of-field in step sizes of one axial pulse length to allow for spectral normalization throughout the depth-of-field during IBC estimation. Reference power spectra, S_{ref}(f), were computed in the frequency domain, and center frequency and bandwidth were measured. A theoretical equation was used to estimate the transducer beamwidth (Szabo, T. L. in Diagnostic Ultrasound Imaging: Inside Out Ch. 6, 178-181 (Elsevier Inc. Academic Press, 2014)).

### Backscattered Echo Acquisition

A schematic of the high-frequency ultrasound imaging system is shown in FIG. 1. Backscattered echoes were acquired using a 58-MHz, single-element, focused, immersion transducer that scanned across the target tissue (i.e., either along the sagittal plane of murine tail or across a liver lobe). Transducer positioning was controlled using the goniometer and 3-axis positioner guided by a custom Matlab® program. The pulser-receiver triggered the firing of the transducer at a pulse repetition frequency of 200 Hz and amplified backscattered echoes, with a receive gain of 50 dB for display on the oscilloscope with a sampling frequency of 2.5 GHz. Each A-line was an average of 10 backscattered echoes to improve the signal to noise ratio (SNR). A-lines were acquired in 60-μm steps, which were greater than one 43-μm beamwidth to ensure independence between neighboring A-lines. Following A-line acquisition, B-mode images displaying acoustic intensity were generated to provide sagittal cross-sectional images of tissue samples. Lateral scans of two planes separated transaxially by 120 μm (i.e., 2 step sizes) were performed for each sample to ensure independence between scatterers in neighboring planes. Thus, scatterers from one resolution cell volume would not interfere with scattering from a resolution cell volume in a neighboring plane. Backscattered echoes were collected as a function of insonification angle for 31 angles ranging from 73°-109° relative to normal incidence in 1° increments, as depicted in FIG. 1. The insonation angle was defined as the angle between the incident ultrasound pulse and the skin surface. This range of angles was selected based on results of pilot experiments.

### Integrated Backscatter Coefficient Estimation

Each backscattered echo was segmented into sub-windows that were 55-μm axially by 60-μm laterally. A Hanning window extracted echo segments in each sub-window in the time domain to reduce ringing in power spectra, and Hanning windows were overlapped by 50% to compensate for signal suppression near window edges (Libgot-Calle, R. et al. Ultrasound Med Biol 34, 252-264 (2008)). Edge-detection algorithms were designed to identify the surfaces of liver, skin, and tendon samples using two-dimensional Gaussian lowpass filtering, median filtering, and intensity thresholding. The remaining quantitative analyses were performed in the frequency domain. The power spectrum of each backscattered echo segment, S(f), was computed and then divided by the transfer function of the receive system at each axial depth, S_{ref}(f), to account for the frequency response of the receive system. Next, a correction was made to compensate for acoustic attenuation in tissue as a function of depth and frequency. Equations for normalized power can be written as follows for liver, W_{L}(f), skin, W_{S}(f), and tendon, W_{T}(f):

$${W}_{L} ( f ) = \frac{S ( f )}{{S}_{r e f} ( f )} {e}^{4 Δ {x}_{L} ( {α}_{L} - {α}_{r e f} )} \qquad ( 1 )$$

$${W}_{S} ( f ) = \frac{S ( f )}{{S}_{r e f} ( f )} {e}^{4 Δ {x}_{S} ( {α}_{S} - {α}_{r e f} )} \qquad ( 2 )$$

$${W}_{T} ( f ) = \frac{S ( f )}{{S}_{r e f} ( f )} {e}^{4 {d}_{S} ( {α}_{S} - {α}_{r e f} )} {e}^{4 Δ {x}_{T} ( {α}_{T} - {α}_{r e f} )} \qquad ( 3 )$$

where Δx_{L}, Δx_{S}, and Δx_{T} are propagation depths into liver, skin, and tendon, respectively. The skin thickness is denoted d_{S}. Attenuation coefficients of liver, skin, tendon, and reference medium are denoted α_{L}, α_{S}, α_{T}, and α_{ref}, respectively. Frequency-dependent attenuation coefficients were expressed as ∝_{L} (f)=0.4f (dB cm⁻¹), ∝_{S} (f)=0.264f^{1.69} (dB cm⁻¹), ∝_{T} (f)=2.42f+3.90 (dB cm⁻¹), and ∝_{ref} (f)=0.0022f² (dB cm⁻¹) (Garcia, T. et al. Ultrasound Med Biol 29, 1787-1797 (2003), Parker, K. J. Ultrasound Med Biol 9, 363-369 (1983), Moran, C. M. et al. Ultrasound Med Biol 21, 1177-1190 (1995), Pinkerton, J. M. M. Proceedings of the Physical Society. Section B 62 (1949)).

The backscatter coefficient, BSC(f), and IBC were computed following power spectra normalization. The BSC(f) is the ratio of backscattered to incident intensity, per unit volume of tissue and is expressed as^{51, 52}

$$B S C ( f ) = \frac{1.45 W ( f ) {R}^{2}}{{A}_{0} Δ z} \qquad ( 5 )$$

where A₀ is the transducer's aperture area, R is the distance between the transducer and the top of the region-of-interest (ROI), and Δz is the depth of the ROI (Mercado, K. P. et al. Ann Biomed Eng 42, 1292-1304 (2014), Cobbold, R. S. C. in Foundations of Biomedical Ultrasound Ch. 5, 270-271 (Oxford University Press, 2007), Insana, M. F. & Hall, T. J. Ultrason Imaging 12, 245-267 (1990)). The IBC is expressed as:

$$IBC = {\int }_{{f}_{min}}^{{f}_{max}} \frac{B S C ( f )}{{f}_{max} - {f}_{min}} d f \qquad ( 6 )$$

where fₘᵢₙ and fₘₐₓ are the minimum and maximum frequencies of the transducer bandwidth (−6 dB), respectively (Mercado, K. P. et al. Ann Biomed Eng 42, 1292-1304 (2014)).

### Quantification of the IBC as a Function of Insonification Angle

Custom Matlab® software was developed to estimate the IBC in murine tissue samples at multiple insonation angles. ROIs with dimensions of 1 mm laterally, by 250 μm, 200 μm, or 55 μm axially, were used to estimate the IBC for tendon and liver, skin, and tendon fascicles, respectively. A ROI width of 1 mm provided 16 A-lines for analyses per ROI. The ROI remained within the theoretical depth-of-field of the transducer as the insonation angle changed. An axial depth of 250 μm allowed 4.5 pulse lengths to be analyzed in each ROI. ROIs were divided into subwindows during the IBC estimation process. IBC subwindows were 60 μm laterally (i.e., 1 step size) and 55 μm axially (i.e., the axial pulse length).

The IBC averaged over the ROI, IBC_{ROI}, was computed following calculation of the BSC(f) and IBC for each subwindow. This process was repeated for 31 insonation angles. Gaussian functions were fitted to curves of IBC versus insonation angle for tendon using the Matlab® curve fitting toolbox. The magnitude of the Gaussian fit was normalized by the number of fascicle edges observed per ROI. Normalized Gaussian fits for two planes separated by 120 μm were averaged for each sample, yielding I̅B̅C̅_{N}. IBC estimates for liver and skin were not modeled by a Gaussian function, thus IBC calculations for two planes separated by 120 μm were averaged for each sample, yielding IBC_{ROI}.

IBC values were computed for tendons of young, wild-type mice (10-16 weeks, n=8), young, diabetic mice (10-16 weeks, n=7), and aged, wild-type mice (20-25 months, n=9). Three additional quantitative metrics based on IBC measurements were defined as parameters for quantitative ultrasound tendon characterization. These metrics provided quantitative parameters associated with scattering strength and angular dependence of scattering strength. FIG. 2 provides an illustration of how these metrics were derived from IBC values. FIG. 2A illustrates I̅B̅C̅_{N} versus insonification angle and two quantitative metrics, I̅B̅C̅_{N,max} and ΔI̅B̅C̅_{N}. The maximum value of I̅B̅C̅_{N}, defined as I̅B̅C̅_{N,max}, provided a metric of backscatter strength. The change in amplitude of I̅B̅C̅_{N} within 10° of I̅B̅C̅_{N,max}, defined as ΔI̅B̅C̅_{N}, provided a metric of the angular dependence of scattering. The derivative of I̅B̅C̅_{N} as a function of insonation angle, θᵢ, was defined as

$$\frac{d {\overline{IBC}}_{N}}{d {θ}_{i}} .$$

FIG. 2B illustrates a plot of

$$\frac{d {\overline{IBC}}_{N}}{d {θ}_{i}}$$

as a function of insonation angle. The linear rate of change of the derivative of the IBC as a function of insonation angle was defined as, M, and was an additional metric to characterize the angular dependence of the IBC near normal incidence. In summary, I̅B̅C̅_{N,max} provided a metric of maximum backscatter strength, and ΔI̅B̅C̅_{N} and M provided metrics of the angular dependence of the IBC.

### Statistical Analyses

Quantitative IBC metrics for murine tail tendon were expressed as mean±standard error of the mean (SEM) and were computed for young, wild-type (n=8), young, diabetic (n=7), and aged, wild-type (n=9) mice. The Grubbs' test was used to identify outliers (Prism, Version 9, GraphPad, La Jolla, CA). A one-way ANOVA was performed followed by Dunnett's multiple comparison test in Prism for each IBC metric. P-values below 0.5 were considered statistically significant.

The results are now described.

### Transducer Characteristics

Transducer characteristics are shown in FIG. 3. The focal distance was measured to be 5 mm. A representative impulse response is shown in FIG. 3A. This transducer emitted a pulse with a wavelength and pulse length measured to be 27 μm and 55 μm, respectively. The depth-of-field (−6 dB) was measured to be 396 μm. The transducer's reference power spectrum is shown in FIG. 3B. The center frequency and −6 dB bandwidth were 58 MHz and 55 MHz, respectively. A theoretical calculation yielded a 43-μm beamwidth (−6 dB).

### IBC Estimation in Tendon

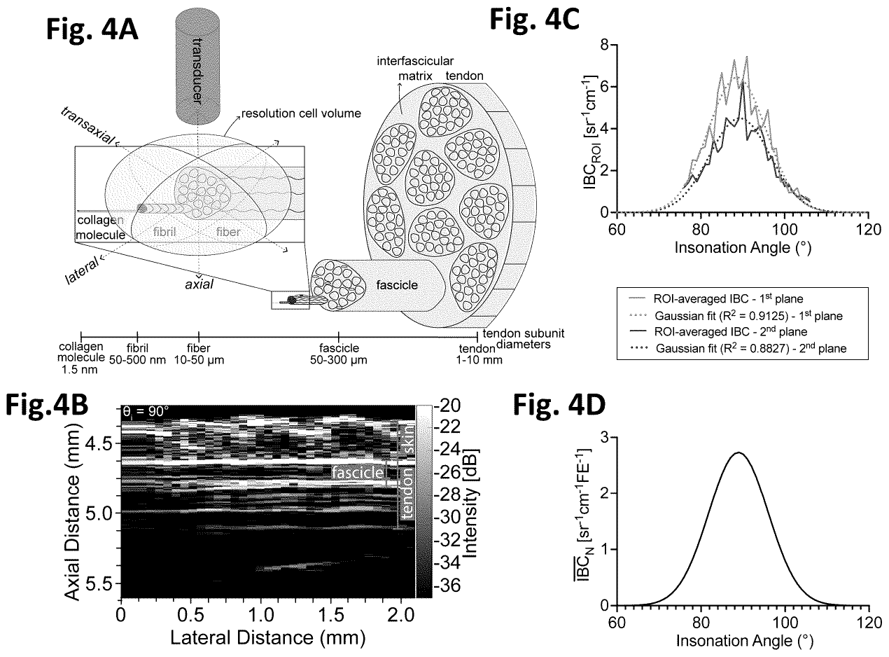

Backscattered echoes are comprised of reflections from different types of scatterers in tissue. These scattering classes are responsible for the different features we observe in conventional B-mode images, including bright edges and speckle. Scatterers are classified based on how their radius, a, compares to the wavelength of the interrogating ultrasound pulse (Szabo, T. L. et al. Diagnostic Ultrasound Imaging: Inside Out Ch. 9, 296-299). Tissue subunits are classified as specular scatterers if they have radii that are much larger than the wavelength of the transducer (ka>>1), and do not fit within the resolution cell volume. For example, specular echoes occur at the surface of skin, tendon, and fascicles imaged at 58 MHz (FIG. 4A). Diffractive scatterers have diameters that are less than a wavelength (ka<1), and there is one or less scatterer per resolution cell volume. For example, diffractive scattering may occur at fascicle and fiber levels in tendon imaged at 58 MHz (FIG. 4A). Diffusive scatterers have diameters that are much less than a wavelength (ka<<1), and there are more than 25 scatterers per resolution cell volume. This type of scattering is responsible for the speckle observed in ultrasound B-mode images. For example, diffuse scattering may occur from collagen fibrils in tendon imaged at 58 MHz (FIG. 4A). A B-mode image showing a sagittal plane of a young, female, wild-type murine tail is shown in FIG. 4B.

A representative example of IBC estimation as a function of insonation angle for a tendon from a young, wild-type mouse is shown in FIG. 4C-D. The average IBC for a 1-mm×250-μm ROI, IBC_{ROI}, at insonation angles of 76°-106° for two 2 planes separated by 120 μm transaxially is shown in FIG. 4C. Gaussian curves were fit to IBC_{ROI} data and R² values were greater than 0.75 for all planes for tendons from young, wild-type mice, indicating that a Gaussian function provides a good model of these IBC data. The IBC_{ROI} normalized by number of fascicle edges and averaged over two planes, I̅B̅C̅_{N}, is shown as a function of insonation angle in FIG. 4D. The I̅B̅C̅_{N} was greatest for insonation angles near normal incidence and decreased for angles above and below normal incidence. This angular dependence of I̅B̅C̅_{N} was observed in all measurements of tendon from young, wild-type mice.

### IBC is Angular Dependent in Tendon and Angular Independent in Skin and Liver

IBC calculations were performed for young, wild-type, female tendon (n=4), overlying tail skin (n=4), and liver (n=4) to test the hypothesis that the IBC as a function of insonation angle, measured with the 58-MHz ultrasound imaging system, can be used to detect collagen fiber alignment in tendon. For each tissue sample, backscattered echoes were acquired, and B-mode images were generated with image dimensions of 2.0 mm (35 A-lines) laterally and 1.3 mm (24 pulse lengths) axially. IBC values were calculated for an ROI of 1 mm×250 μm for tendon and liver, and for an ROI of 1 mm×200 μm for skin. Translucent parametric images of IBC values were then overlayed onto B-scan images.

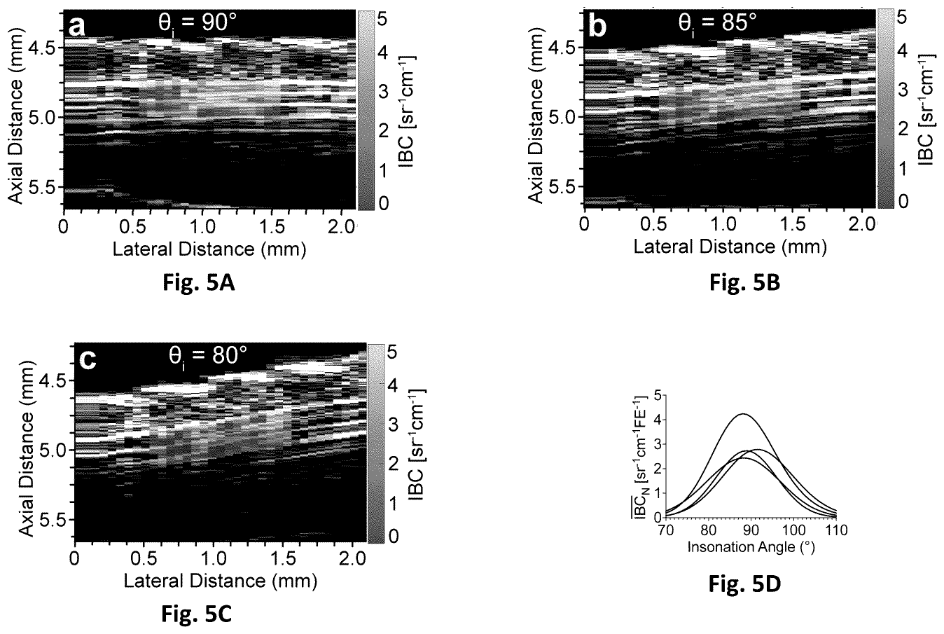

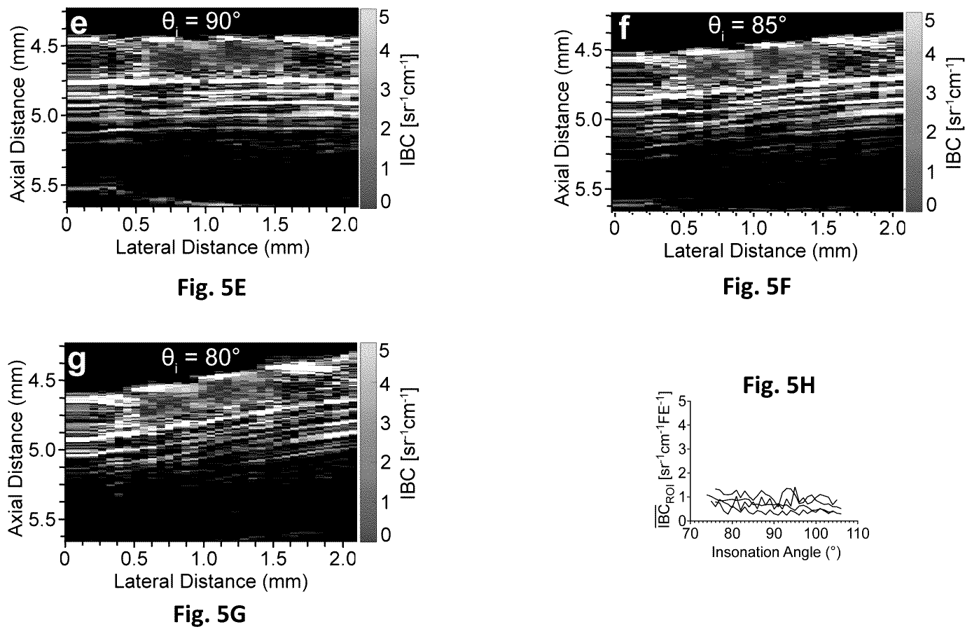

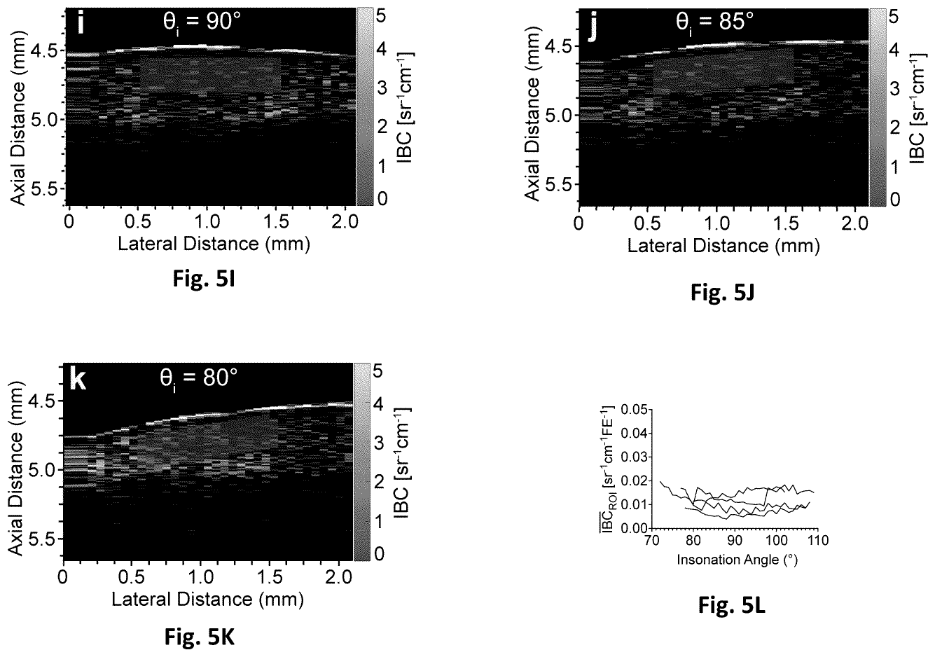

FIG. 5 presents results of IBC measurements as a function of insonation angle for wild-type tendon (FIGS. 5A-D), skin (FIGS. 5E-H), and liver (FIGS. 5I-L). FIGS. 5A-C presents B-mode and IBC images at select insonation angles of 90°, 85°, and 80° for young, wild-type tendon. In each image, the top echo arises from the interface between the PBS and the skin surface. The thickness of the skin was ˜300 μm. Bright echo lines below the skin indicate the edges of tendon fascicles and fibers. The strongest backscatter occurred at fascicle edges within tendons. ROIs of dimension 1 mm×250 μm were used to calculate IBC of tendon. As insonation angle changed from 90° to 85° to 80°, the magnitude of IBC within the ROI decreased, as evidenced by the decrease in yellow pixels and increase in green and blue pixels within the ROIs (FIG. 5A-C). FIG. 5D presents I̅B̅C̅_{N} as a function of angle for all 31 angles of incidence for tail tendons for all mice tested (n=4). The magnitude of I̅B̅C̅_{N} was maximum near normal incidence and decreased for angles greater than or less than normal incidence (FIG. 5D).

FIGS. 5E-G present representative B-mode and IBC parametric images at insonation angles of 90°, 85°, and 80° for overlying tail skin from young, wild-type mice. B-mode images indicated that scattering within skin was more heterogeneous compared to tendon. ROIs (1 mm×200 μm) for estimation of IBC were located within the skin region. IBC parametric images were similar as insonation angle changed from 90° to 85° to 80° (FIGS. 5E-G). FIG. 5H presents IBC_{ROI} as a function of insonation angle for skin for all mice tested (n=4). The IBC_{ROI} for skin did not exhibit an angular dependence for any mice (FIG. 5H). Thus, subsequent processing to fit Gaussian curves to the data and compute I̅B̅C̅_{N} was not performed for skin regions.

FIGS. 5I-K present representative B-mode and IBC parametric images at insonation angles of 90°, 85°, and 80° for livers from young, wild-type mice. In each image, the top echo arises from the interface between the PBS and the liver surface. B-mode images indicated that scattering within liver was lower in amplitude and more uniform compared to both skin and tendon. ROIs (1 mm×250 μm) for IBC calculations were located ˜50 μm beneath the liver surface. FIG. 5L presents IBC_{ROI} data as a function of insonation angle for liver for all mice tested (n=4). IBC_{ROI} for liver did not exhibit an angular dependence for any mice (FIG. 5L) and, thus, subsequent processing to fit Gaussian curves to the data and compute I̅B̅C̅_{N} were not performed for liver regions.

In summary, B-mode, IBC images, and IBC parameters as a function of angle were generated for murine tail tendon, overlying tail skin, and liver in wild-type mice. As expected, backscatter from tendon was greater than from skin or liver. In young, healthy, wild-type mice, the IBC was dependent on insonation angle in tendon, while for skin and liver, the IBC was independent of the angle of insonation.

### IBC is Angular Dependent at the Intratendon and Intrafascicular Levels

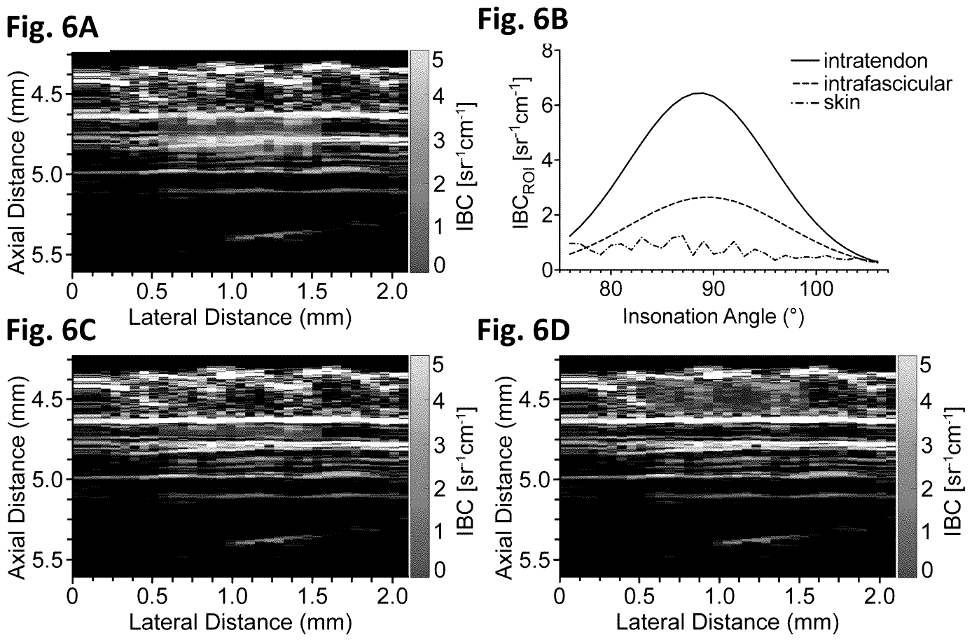

A single tendon is comprised of a hierarchy of parallel cylindrical subunits of decreasing diameter, namely fascicle (˜50-300 μm diameter), fiber (˜10-50 μm diameter), fibril (˜50-500 nm), and collagen molecule (˜1.5 nm) (Voleti, P. B. et al. Annu Rev Biomed Eng 14, 47-71 (2012)). The objective of this study was to test whether the IBC was angular dependent for weaker sub-resolution scatterers in ROIs located within individual fascicles (i.e., intrafascicular). FIG. 6A presents representative B-mode and IBC parametric images for an intratendon ROI (1 mm×250 μm) that encompassed tendon fascicle edges and intrafascicular regions. As observed in FIG. 6A, scattering from tendon edges was greater than scattering from the intrafascicular region. The IBC_{ROI} as a function of angle for data in FIG. 6A exhibited an angular dependence (FIG. 6B solid blue line). FIG. 6C presents representative B-mode and IBC parametric images for a smaller ROI (1 mm×55 μm) that encompassed only the intrafascicular region. The dashed blue line in FIG. 6B represents the IBC_{ROI} as a function of angle for the intrafascicular ROI shown in FIG. 6C. For the intrafascicular region, the IBC_{ROI} was also dependent on angle of incidence, although the overall magnitude of the IBC_{ROI} was less than that of ROIs from intratendon regions. For comparison, FIG. 6D and the solid black line in FIG. 6B present comparable data for a ROI (1 mm×200 μm) in skin. As in previous results, the IBC_{ROI} of skin was not dependent on angle and the magnitude of the IBC_{ROI} was less than that of the intratendon ROI, even though the ROI dimensions were comparable to that of FIG. 6A.

### IBC Detects Differences in Collagen Microstructure in Tendon in the Presence of Diabetes and Aging

Diabetes and aging are associated with alterations in tendon microstructure (Batista, F. et al. Foot Ankle Int 29, 498-501 (2008), Boivin, G. P. et al. Muscles Ligaments Tendons J 4, 280-284 (2014), Studentsova, V. et al. Sci Rep 8, 9218 (2018), Ackerman, J. E. et al. J Orthop Res (2017)). Thus, it was tested whether metrics associated with the angular dependence of the IBC could detect differences in tendon between wild-type, diabetic, and aged mice. B-mode images were generated as a function of insonation angle and IBC calculations were performed for young, wild-type (n=8), aged, wild-type (n=9), and young diabetic (n=7) female, tail tendon (FIG. 7).

Shown in FIGS. 7A-C are representative B-mode and IBC parametric images for young, wild-type (FIG. 7A), aged, wild-type (FIG. 7B), and young, diabetic (FIG. 7C) tail tendon. B-mode images qualitatively suggested fascicle disorganization with aging and in the presence of diabetes compared to tendon from young, wild-type mice. In support of this qualitative assessment, quantitative IBC parametric images indicated a decrease in scattering strength for aged, wild-type tendon (FIG. 7B) and young, diabetic tendon (FIG. 7C) compared to young, wild-type tendon (FIG. 7A), as evidenced by the increase in blue and green pixels compared to yellow and orange pixels.

Representative plots of I̅B̅C̅_{N} as a function of insonification angle are shown for tail tendons from young, wild-type (FIG. 7D), aged, wild-type (FIG. 7E), and young, diabetic (FIG. 7F) female mice. To quantify differences in the magnitude and angular dependence of the I̅B̅C̅_{N}, three quantitative ultrasound metrics were defined and computed. As described in Materials and Methods, the I̅B̅C̅_{N,max} provided a metric of maximum backscatter strength, and ΔI̅B̅C̅_{N} and M provided metrics of the angular dependence of I̅B̅C̅_{N}.

Data for the quantitative metric, I̅B̅C̅_{N,max}, are shown in FIG. 7G. The I̅B̅C̅_{N}, max (±SEM) of tendons from young, wild-type mice (3.03±0.33 sr⁻¹ cm⁻¹ FE⁻¹) was significantly greater than that of both young, diabetic mouse tendons (1.55±0.25 sr⁻¹ cm⁻¹ FE⁻¹) (p=0.0029) and aged, wild-type mouse tendons (1.92±0.25 sr⁻¹ cm⁻¹ FE-1) (p=0.019).

As shown in FIG. 7H, the average ΔIBC_{N} (±SEM) of tendons from young, wild-type mice (0.73±0.11 sr⁻¹ cm⁻¹ FE⁻¹) was significantly greater than that of both young, diabetic mouse tendons (0.33±0.055 sr⁻¹ cm⁻¹ FE⁻¹) (p=0.0062) and aged, wild-type mouse tendons (0.31±0.063 sr⁻¹ cm⁻¹ FE⁻¹) (p=0.0027). Similarly, the mean value of M (±SEM) of tendons of young, wild-type mice (−196±33 sr⁻² cm⁻¹ FE⁻¹) was significantly greater than that of both young, diabetic (−87±15 sr⁻² cm⁻¹ FE⁻¹) (p=0.0085) and aged, wild-type mouse tendons (−77±16 sr⁻² cm⁻¹ FE⁻¹) (p=0.0034). In summary, all three quantitative IBC metrics (I̅B̅C̅_{N,max}, ÄI̅B̅C̅_{N}, M) were significantly different in tendons from aged, wild-type and young, diabetic mice compared to young, wild-type mice.

Taken together, these data demonstrate the utility of three quantitative IBC metrics for discerning differences in tendon microstructure in the presence of diabetes and with age. All three quantitative IBC metrics (I̅B̅C̅_{N,max}, ΔI̅B̅C̅_{N}, M) were significantly different in tendons from diabetic mice and aged mice compared to tendons from wild-type mice.

The Example summary is now described.

A quantitative high-frequency, ultrasound technique based on the IBC was advanced to characterize tendon microstructure non-invasively and non-destructively. Using a 58-MHz ultrasound system, backscattered echoes as a function of insonation angle were obtained from murine tail tendon and compared to overlying tail skin, and liver. The imaging characteristics of this transducer enabled resolution of tendon, fascicles, and collagen fibers with a diameter greater than the interrogating pulse wavelength, while smaller collagen fibers and fibrils contributed to sub-resolution scattering. B-mode and IBC parametric images were generated at each insonation angle and averages of IBC metrics for a ROI were computed. The IBC exhibited a clear angular dependence in tendons with aligned collagen fibers. In tendons from young, wild-type mice, peak values of IBC_{ROI} and I̅B̅C̅_{N} were observed near normal incidence, and magnitudes of these parameters decreased as angles moved away from normal incidence (FIGS. 4 and 5). In comparison, the IBC was not dependent upon insonation angle for more heterogeneous tissues, as observed with liver and tail skin (FIG. 5). Magnitudes of I̅B̅C̅_{ROI} and I̅B̅C̅_{N} were greater in tendon compared to skin and liver. In tendon, magnitudes of the IBC were greatest at fascicle edges that are organized in a parallel arrangement within tendon. Interestingly, the angular dependence of the IBC was observed when ROIs included multiple fascicles (intratendon), as well as when the IBC was evaluated using smaller ROIs that were within a single fascicle (intrafascicular) (FIG. 6). Thus, weaker, sub-resolution scattering within fascicles (i.e. likely arising from fibers and fibrils) also exhibited an angular dependence of the IBC.

The feasibility of employing the angular dependence of the IBC to detect alterations in tendon microstructure in the presence of diabetes and aging was investigated. Three quantitative ultrasound metrics derived from the magnitude and angular dependence of the IBC were defined and tested for their ability to quantitatively characterize differences in tendon microstructure between tendon from young, wild-type mice, tendon from young, diabetic mice, and tendon from aged, wild-type mice. These metrics were the (1) I̅B̅C̅_{N}, max, the maximum IBC, (2) ΔI̅B̅C̅_{N}, the change in the IBC within ten degrees of its maximum, and (3) M, the linear rate of change of the derivative of the IBC as a function of insonation angle. The I̅B̅C̅_{N,max} provided a metric to quantify the magnitude of the backscatter, while ΔI̅B̅C̅_{N} and M parameters provided metrics to characterize the angular dependence of the backscatter.

All three quantitative ultrasound metrics were significantly lower in tendon from diabetic mice compared to tendon from wild-type mice, indicating that scattering strength was reduced, and tendon disorganization was increased in diabetic tendon compared to wild-type tendon (FIG. 7). These findings with diabetic murine tendon are consistent with a prior study that qualitatively visualized tendon disorganization and localized hypoechoic regions in clinical B-mode ultrasound images of female diabetic Achilles tendon (Abate, M. et al. Foot Ankle Int 35, 44-49 (2014), Afolabi, B. I. et al. J Ultrason 20, e291-e299 (2021)). Using qualitative B-mode ultrasound imaging, tendon disorganization has even been observed in diabetic patients with no symptoms of tendinopathy, suggesting the potential relevance for new technologies to detect tendon disorganization before patients are symptomatic (Batista, F. et al. Foot Ankle Int 29, 498-501 (2008)). Furthermore, in patients with a normal body mass index, tendon disorganization was significantly higher in patients with diabetes compared to non-diabetic patients (Abate, M. et al. Foot Ankle Int 35, 44-49 (2014)). Moreover, tendinopathy remained when metabolic changes associated with diabetes were reversed in mice, highlighting the importance of monitoring diabetic tendon structure under effective glycemic management (Studentsova, V. et al. Sci Rep 8, 9218 (2018)).

The ability of this quantitative ultrasound technique to detect differences in tendon microstructure alignment in aged murine tendon was demonstrated. The parameters, I̅B̅C̅_{N,max}, ΔI̅B̅C̅_{N} and M were significantly lower in aged, wild-type mice compared to young, wild-type mice, indicative of an increase in tendon disorganization in aged compared to young tendon (FIG. 7). These results of IBC measurements for aged, wild-type tendon are consistent with studies using histology and polarized light microscopy that reported an increase in collagen fiber disorganization in aged compared to young murine supraspinatus tendons as well as flexor digitorum longus tendons (Ackerman, J. E. et al. J Orthop Res (2017), Connizzo, B. K., et al. J Biomech Eng 135, 021019 (2013)). Furthermore, aging can impair tendon healing in aged compared to young murine tendon, with more disorganized collagen fibers at the injury site of aged mice (Ackerman, J. E. et al. J Orthop Res (2017)). Since collagen disorganization and the potential for tendon rupture increase with age, new non-invasive techniques for characterizing tendon microstructure may be an important component to assessing characteristics of tendon with aging that are not revealed by tendon mechanical properties alone (de Jonge, S. et al., Br J Sports Med 45, 1026-1028 (2011)).

This quantitative ultrasound tissue characterization technique may be used as a pre-clinical tool for tendon research, and as a point-of-care clinical approach for longitudinal monitoring of tendon injury and repair to provide guidance to clinicians and physical therapist on healing and rehabilitation regimens. Intact murine tail tendon was employed for testing as it is a common tendon model with cylindrical geometry that facilitates image acquisition as a function of insonation angle while avoiding unintended strain to the tendon. However, tendon anatomy, crimp, and mechanical properties can vary based on anatomical location (Zuskov, A. et al. J Orthop Res 38, 36-42 (2020)). This technique may be used to quantitatively image additional tendon types, including murine Achilles and flexor tendons which are models often used for studies of tendon injury, disease, and healing. The technique may be used for characterizing tendon microstructure longitudinally in the presence of tendon injury and repair and may be a tool for monitoring tendon injury, degeneration, and healing. This technique may be translated to imaging human tendons, such as Achilles, flexor, or other superficial tendons.

A state-of-the-art, high-frequency transducer and pulser-receiver that were designed specifically for imaging murine tendon was employed. The penetration depth of the 58-MHz transducer can enable quantitative imaging of murine Achilles and flexor digitorum longus tendons, which are ˜200-500 μm and ˜200 μm thick, respectively (Boivin, G. P. et al. Muscles Ligaments Tendons J 4, 280-284 (2014), Wang, P. H. et al. Biomed Opt Express 2, 1462-1469 (2011), Ackerman, J. E. & Loiselle, A. E. J Vis Exp (2016)). This technology may be translated to the clinic. The technique may be used at lower frequencies to enable greater penetration depth. Transducers may be designed to optimize use with specific tendon applications. Array transducers may be implemented for real-time imaging of static or dynamic tendon.

A quantitative, high-frequency ultrasound imaging technique to characterize murine tendon non-invasively and non-destructively was advanced. Metrics associated with the magnitude and angular dependence of the IBC were developed to detect collagen fiber alignment in tendons of young, wild-type mice, and discern alterations in tendon microstructure in tendons of diabetic and aged mice. The utility of this ultrasound technique employing metrics associated with the IBC to characterize tendon microstructure non-invasively was demonstrated. This technique may be used as a pre-clinical tool for tendon research, and as clinical diagnostic approach for longitudinal monitoring of tendon injury and repair in healthy, diabetic, and aged patients.

### Example 2: Quantitative Ultrasound System and Methods for Characterizing Structure of Tissues and Materials Using Array-Based Ultrasound Systems

Methods and techniques described in Example 1 were further expanded through the use of array-based ultrasound transducers. Ultrasound array transducers provide additional technical capabilities to enable translation to clinical and commercial applications, including for example real-time imaging, electronic beam steering, electronic focusing, and rapid volumetric imaging and data acquisition. The IBC as a function of insonation angle, and metrics associated with the IBC, can be used to characterize tissue and material microstructure.

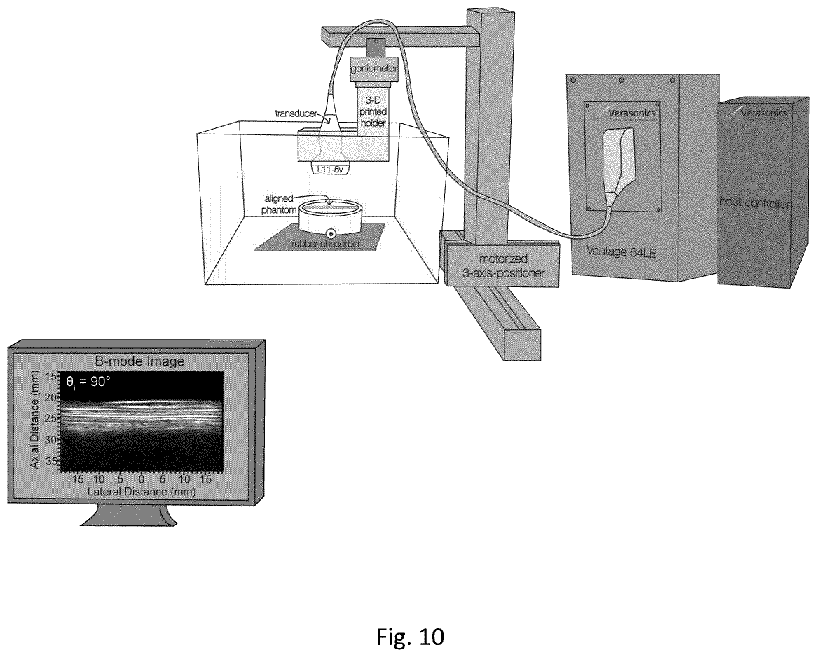

An exemplary embodiment of a quantitative ultrasound imaging system is represented in FIG. 10. A Verasonics Image Acquisition System is represented. An 8-MHz linear array transducer is clamped into a custom 3-D printed holder and guided by a 3-axis-positioner. The transducer is controlled using a Vantage 64LE system, host controller, and custom Matlab program. A phantom comprised of aligned 100-μm diameter polyester surgical suture fiber is submerged in degassed, deionized water. A rubber absorber is placed below the phantom to prevent reverberations.

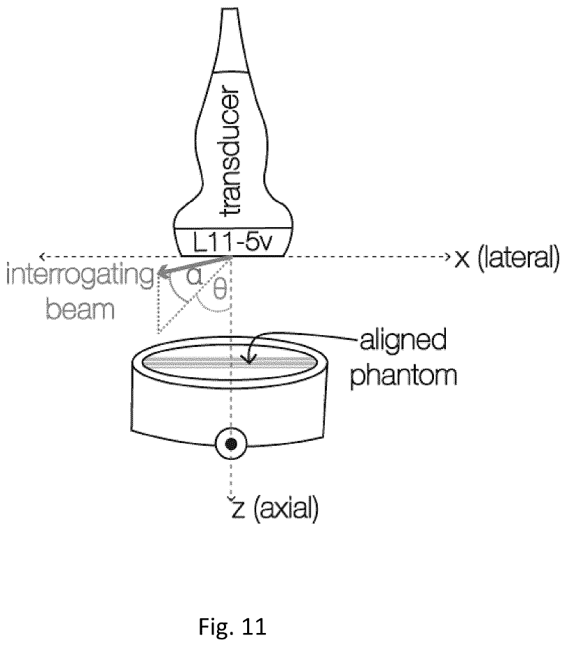

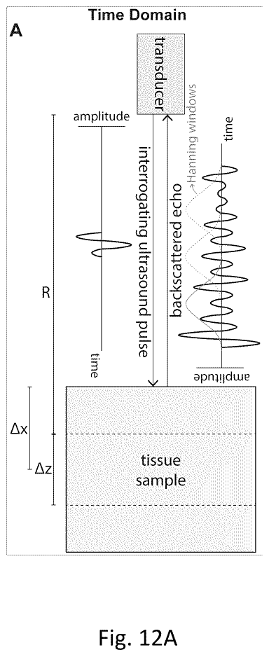

An exemplary embodiment of a beam steering method is presented in FIG. 11. An 8-MHz linear array transducer is positioned above the aligned suture fiber phantom at normal incidence. The interrogating beam position is defined by two angles. The azimuth, θ, is the angle between the beam and z-axis along the xz-plane. The elevation, a, is the angle between the beam and the z-axis along the yz-plane. The elevation was set to 0°, and the azimuth was varied from 90°-85° in 1° increments for the following experiment. Beam steering with a linear array allows for faster image acquisition compared to imaging with a single element transducer and manually varying the insonation angle.

An exemplary embodiment of an IBC estimation process is presented in FIG. 12. A backscattered echo acquired in the time domain is segmented by Hanning sub-windows (FIG. 12A). Each segment is converted into a power spectrum, S(f), in the frequency domain (FIG. 12B). The receive system's transfer function, S_{ref}(f), is divided from S(f) to correct for the frequency response of the receive system (FIG. 12C). Attenuation corrections, A_{S}(f), A_{F}(f), and A_{M}(f), are made for overlying tissues or materials (such as skin, fat, and/or muscle, respectively) yielding a normalized power spectrum, W(f) (FIG. 12D-12E). The BSC(f) and IBC are computed for each sub-window (FIG. 12F) and metrics associated with the IBC are as described previously.

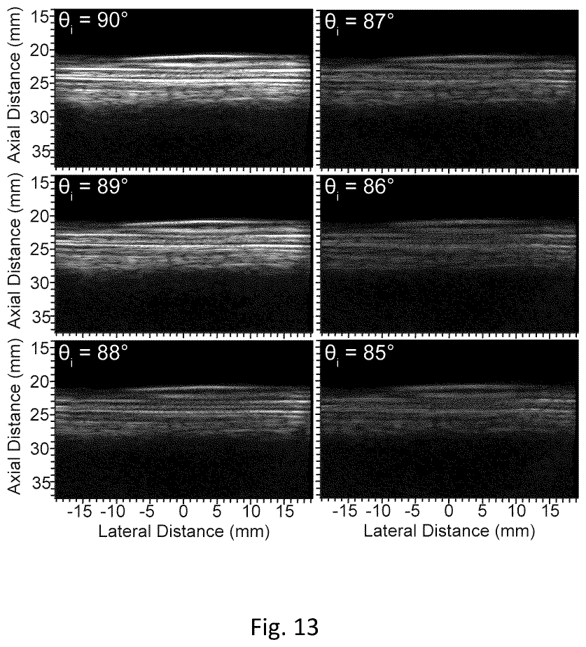

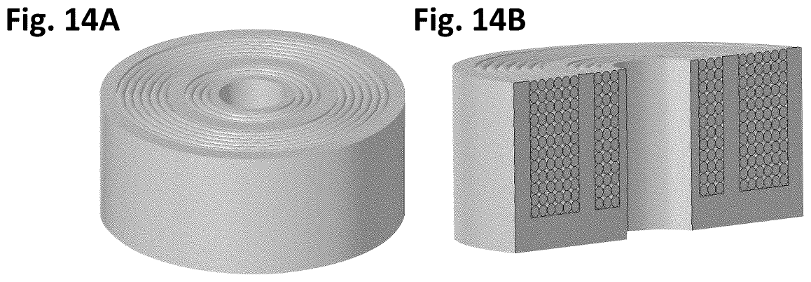

Backscattered echoes and B-mode images of a phantom with aligned fibers were acquired at multiple insonation angles using an 8-MHz linear array transducer (Verasonics). Phantom echogenicity was highest at normal incidence and decreased for angles greater than and less than normal incidence (FIG. 13). Angular dependence of echogenicity was observed for aligned structures at 58-MHz and 8-MHz (FIG. 13). B-mode images of a phantom with microstructural alignment at insonation angles of 90°-85° in 1° increments are shown in FIG. 13. Backscattered echoes were beamformed by Verasonics' proprietary software. Echogenicity exhibited anisotropy in an aligned fiber phantom. FIG. 13 illustrates results of a phantom with linearly aligned microstructure, however, the technique can be further expanded to tissues and materials with other spatial alignments, for example circularly concentric alignment. FIG. 14 depicts an example of a tissue with circularly concentric alignment.

The capabilities of the quantitative ultrasound system and methods were further expanded through the use of a hand-held, point-of-care ultrasound imaging device. In addition to advantages of array-based transducers described above (including real-time imaging, electronic beam steering, electronic focus, and rapid volumetric imaging and data acquisition), hand-held, point-of-care ultrasound imaging devices can offer additional capabilities to enable translation to clinical and commercial practices, including portability, low-cost, commercially available, often FDA approved, imbedded RF capture for data acquisition, volumetric spatial tracking of transducer positioning, and wireless connectivity for image and data acquisition and storage.

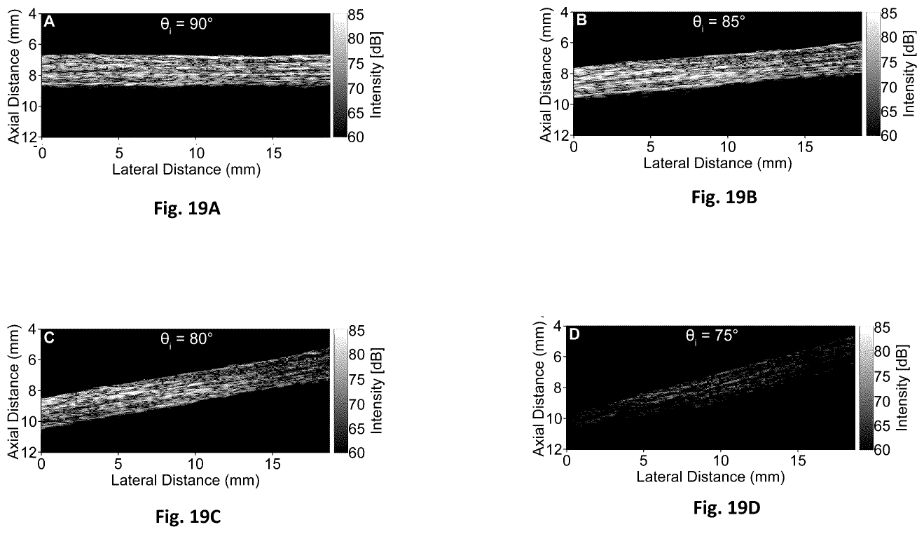

An example is next presented of the quantitative ultrasound system and methods using a point-of-care ultrasound device. The Clarius L20 ultra-high frequency, linear-array ultrasound device was used with transmit frequency of 14 MHz. Backscattered echoes and B-mode images of porcine median nerve were acquired at multiple insonation angles using the Clarius L20 imaging device. Echogenicity of porcine median nerve was highest at normal incidence and decreased away from normal incidence (FIG. 19). In other words, Echogenicity of porcine median nerve is highest at normal incidence and decreases as the insonation angle decreases.

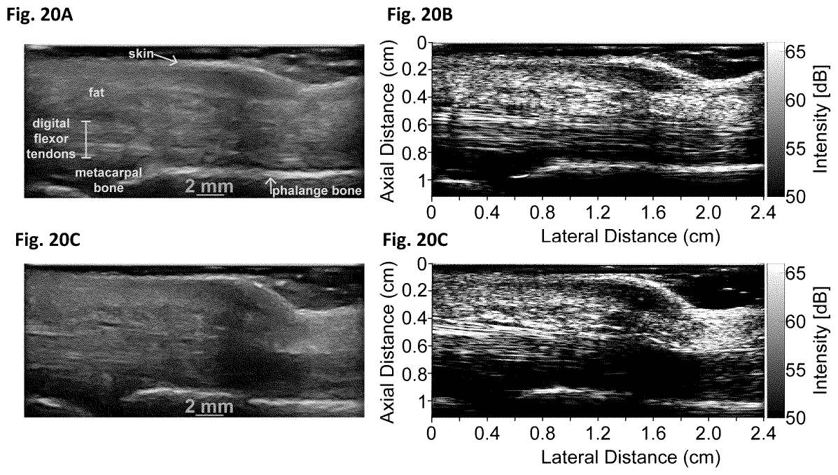

An advantage of array-based transducers is the ability to image and acquire RF data in real-time, particularly during functional movements. The Clarius L20 was also demonstrated to image tissue structure and detect changes in echogenicity in human digital flexor tendon during a functional movement, namely tendon flexion. Digital flexor tendons lie below skin and fat and above bone. The Clarius L20 device was used to image flexor tendon of the human hand at rest (FIG. 20A, B) and during flexion (FIG. 20C, D). The transmit frequency was 14 MHz. FIGS. 20A and 20C depict B-mode images generated with the proprietary software of the Clarius L20. In comparison FIGS. 20B and 20D depict B-mode images computed from raw RF data exported by the Clarius. Microstructural alignment in the tendon is more evident in B-mode images computed from RF data compared to the images displayed directly on the Clarius user device after proprietary Clarius filtering and image processing. Thus, performing quantitative ultrasound techniques on raw backscattered echoes may reveal characteristics that would otherwise be lost during image processing and filtering. Thus, the described quantitative ultrasound techniques that are derived from the raw RF data may be more effective in detecting changes in microstructure or other tissue structure properties. In tests, echogenicity of the flexor tendon was observed to be dependent on angle of insonation and flexion.

In summary, this example demonstrates the extension of the quantitative ultrasound system and methods to implementation with array-based transducers and point-of-care ultrasound imaging devices. Array-based transducers offer numerous capabilities that are advantageous for translation to clinical and commercial applications, including real-time scanning, electronic beam-steering, electronic focusing, and rapid volumetric imaging and data-acquisition. Point-of-care ultrasound imaging devices provide further advantages including portability, low-cost, FDA approval, imbedded RF capture for data acquisition, volumetric spatial tracking of transducer positioning, and wireless connectivity for image and data acquisition. Thus, technical capabilities of array-based transducers and point-of-care imaging devices can enable real-time, quantitative IBC tissue characterization, even during functional movement. As one example, it is envisioned that quantitative IBC characterization of tissues (such as tendon, nerve, ligament, and muscle) as a function of strain (or stress or other mechanical measures) can provide information for clinicians to aid in diagnoses and guide therapeutic and/or rehabilitation regimens.

### Example 3: Quantitative Ultrasound System and Methods for Characterizing Tissue Microstructure of Pelvic Muscles Including the Anal Sphincter and the Puborectalis Muscles

Anorectal malformations are congenital disorders that impair development of the rectum, anal canal, and anal sphincter complex. Following surgical repair during infancy, patients experience continued challenges with fecal continence, which depends on the structure and function of the anal sphincter complex. However, surgeons lack quantitative imaging technologies to assess anal sphincter complex structure.

Spectral analysis techniques allow for characterization of soft tissue microstructure. The integrated backscatter coefficient (IBC) is a quantitative ultrasound metric that provides an estimate of how strongly collagen fibers in tissue scatter sound back to the transducer.

Anorectal malformations are significant congenital disorders that result in structural and functional impairments and occur in 2-5 in 10,000 births (Rollins M D, et al. J Pediatr. 2022 January; 240:122-128). After surgical correction, fecal continence depends, in part, on the structure and function of the anal sphincter complex as tissue microstructure impacts contraction strength. Characterizing the microstructural properties of the anal sphincter complex is crucial for understanding how microstructural changes during development and after surgical repair impact function in patients with anorectal malformations. Furthermore, innovation of an in vivo, non-invasive, quantitative tool for microstructural assessment of the anal sphincter complex will enhance surgical approaches and optimize tissue functional capabilities following surgery.

The quantitative ultrasound system, methods, and spectral analysis techniques were developed to characterize the microstructure of pelvic muscles, such as the puborectalis muscle and anal sphincter complex. Methods and spectral analysis techniques developed in murine tendon for a 58-MHz single-element transducer were expanded to pelvic, including the anal sphincter complex and puborectalis muscle complex, using either a 58-MHz single-element transducer or an 8-MHz linear array transducer, to demonstrate potential for clinical translation of this approach. The IBC as a function of insonation angle may be used to characterize tissue microstructure alignment in pelvic muscles, including puborectalis muscle and anal sphincter complex.

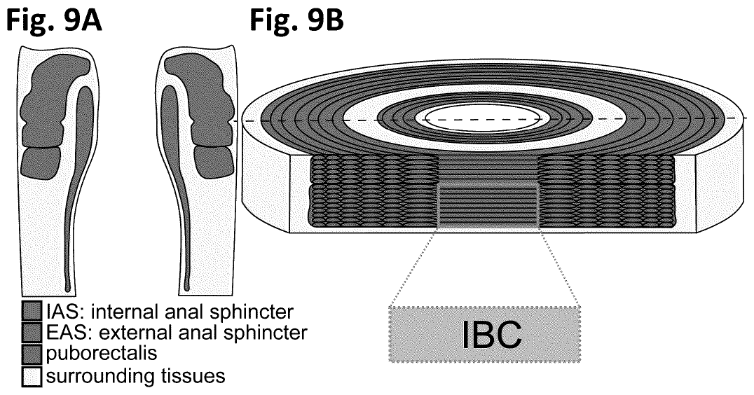

Pelvic muscle anatomy, including the anal sphincter complex and puborectalis muscle, is represented in FIG. 9. Backscatter data as a function of insonation angle were acquired as described previously, and used to generate B-scan images, IBC parametric images, and IBC metrics. Following backscattered echo acquisition, IBC estimates were gathered from region-of-interests within either the anal sphincter complex or the puborectalis muscle. The sagittal plane of the perineum including the internal anal sphincter, external anal sphincter and puborectalis muscle (grey) are represented in FIG. 9A. A schematic of a 3-D slice of the perineum, including a region-of-interest within the external puborectalis where IBC estimates can be performed following backscattered echo acquisition is represented in FIG. 9B.

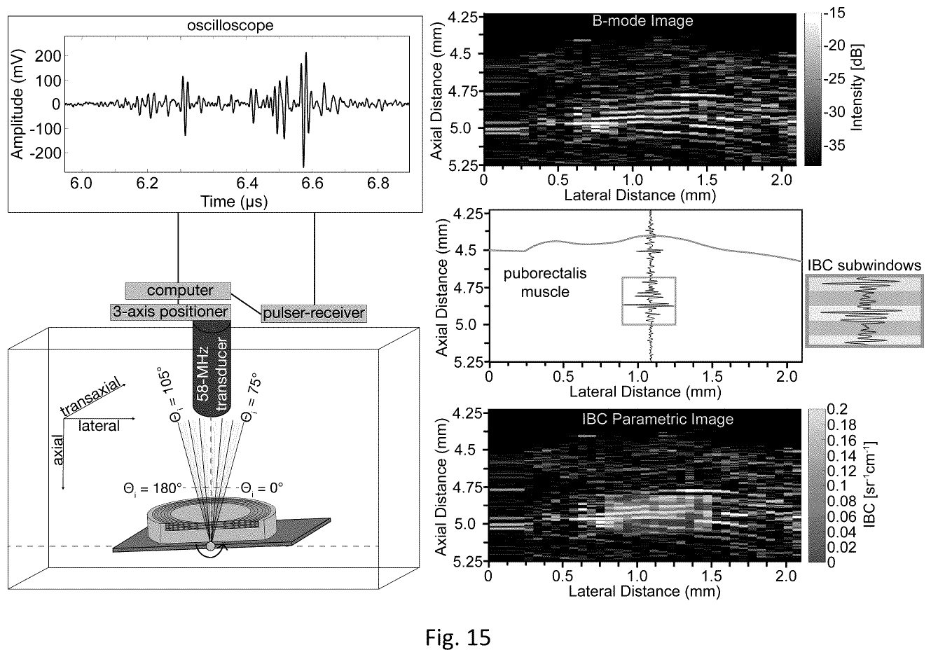

The high-frequency ultrasound imaging system described in example 1 was adapted for quantitative imaging and tissue characterization of pelvic muscles and is presented in FIG. 15. Briefly, a 58-MHz transducer was used to acquire backscatter signals as a function of insonation angle near normal incidence. Backscatter data were then used to generate B-mode images, IBC metrics, and IBC parametric images as described in example 1. The technique was demonstrated with rabbit anal sphincter muscle and puborectalis muscle ex vivo.

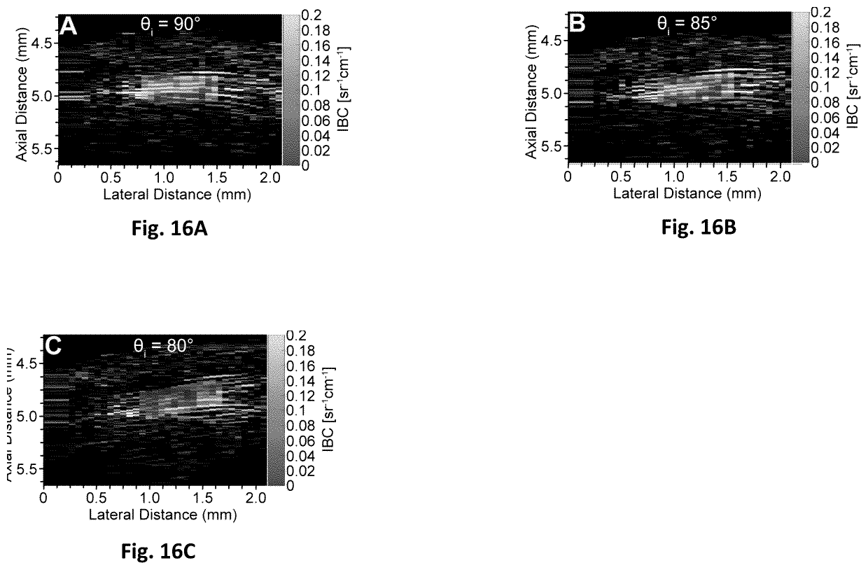

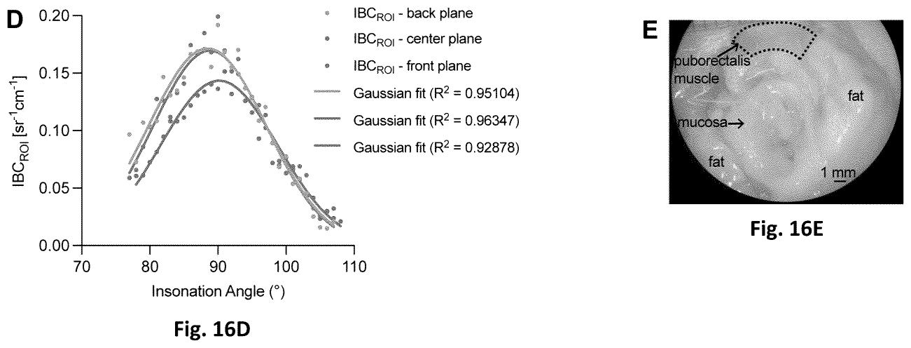

The IBC as a function of insonation angle can be used to detect microstructural alignment in healthy rabbit puborectalis muscle ex-vivo. Representative B-mode images of healthy rabbit puborectalis muscle are shown in FIG. 16. Microstructure of puborectalis muscle is characterized by fiber alignment. IBC parametric images of healthy rabbit puborectalis muscle ex vivo were generated at multiple insonation angles (FIG. 16). IBC measurements of one imaging plane of healthy rabbit puborectalis are shown at 3 representative angles of insonation (90, 85, 80 degrees). The average IBC in a region-of-interest were plotted as a function of insonation angle for three imaging planes separated by one beamwidth to ensure independence between neighboring scatterers (FIG. 16). The IBC was strongly dependent on insonation angle in the puborectalis muscle. Magnitude of the IBC was greatest near normal incidence and decreased at angles greater than and less than normal incidence.

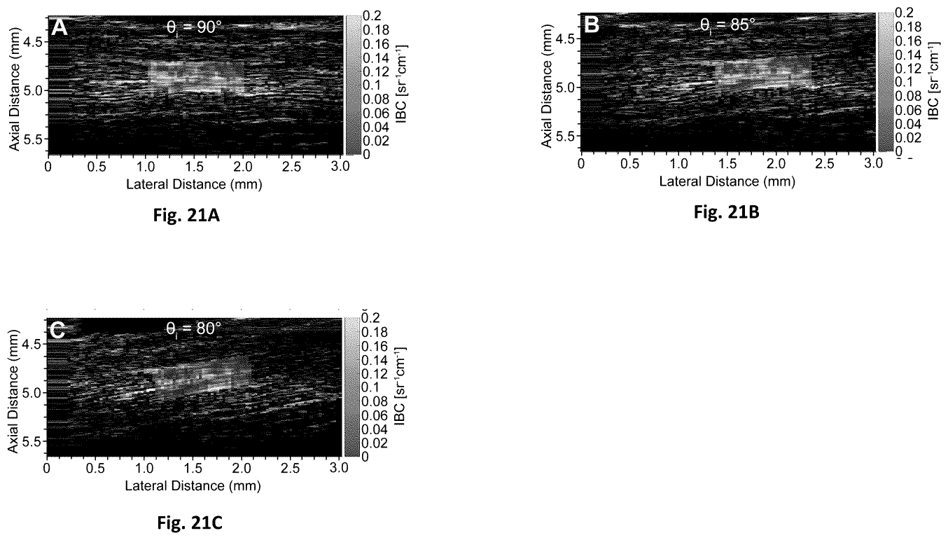

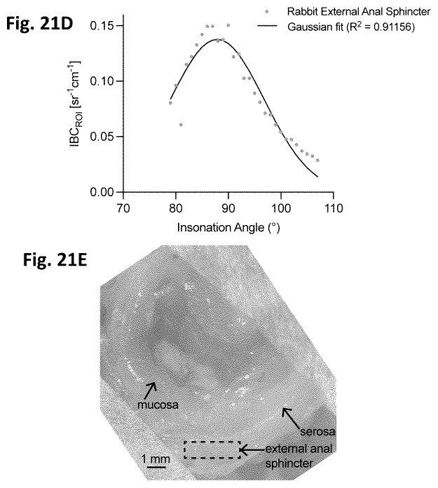

The quantitative ultrasound system and methods were also demonstrated in the rabbit anal sphincter. Using the 58-MHz system, backscatter data was acquired as a function of insonation angle in rabbit anal sphincter ex vivo and metrics associated with the IBC were generated as described previously. FIG. 21A-C show B-scan images and overlayed IBC parametric images for rabbit anal sphincter at 3 representative angles of insonation (90, 85, 80 degrees). FIG. 21D presents the average IBC in a ROI as a function of all insonation angles (data points) and a Gaussian fit to the data (solid curve). Maximum IBC occurs near normal incidence and IBC magnitude decreases for angles above and below normal incidence. This example demonstrates the capability of the technique in tissues with circularly aligned microstructure (FIG. 21E). Alterations in microstructure of the anal sphincter muscle due to disease, injury, or malformation would result in decreased in the angular dependence of the IBC and associated quantitative metrics.

In summary, a high-frequency quantitative ultrasound system was developed to measure the integrated backscatter coefficient (IBC) as a function of insonation angle in rabbit anal sphincter and puborectalis muscle ex vivo. Microstructural tissue alignment in anal sphincter and puborectalis muscle resulted in a dependence of the magnitude of IBC as a function of insonation angle. In comparison, the IBC was independent of angle in homogenous liver tissue. Changes in microstructural fiber alignment in anal sphincter or puborectalis muscle will diminish the angular dependence of the IBC. Thus, the system and methods can be used to image, detect, and quantify changes in tissue microstructure resulting from, for examples disease, injury, or malformations that affect microstructure in pelvic muscles such as the anal sphincter and puborectalis muscles.

### Example 4: Quantitative Ultrasound System and Methods for Characterizing Tissue Microstructure of Nerve

Neural tissue is also characterized by microstructure tissue alignment, and a hierarchical microstructure where a single nerve is comprised of multiple fascicles each of which are comprised of multiple axons. Similarly to other tissues, microstructural alignment of nerve can be affected by injury and disease. Diabetic patients are over three times more likely to develop tendinopathy than non-diabetic patients and ˜50% of diabetic patients have neuropathy. Diabetes also impairs nerve regeneration and the chance of nerve injury increases with age. Thus, in this example a quantitative ultrasound system and methods to characterize tissue microstructure of nerve is demonstrated.

The quantitative ultrasound system, methods, and spectral analysis techniques developed in tendon were expanded for application to nerve. Briefly, the 58-MHz transducer system was used to acquire backscatter signals from porcine median nerve ex vivo as a function of insonation angle above and below normal incidence. Backscatter data were then used to generate B-mode images, IBC metrics, and IBC parametric images as described previously.

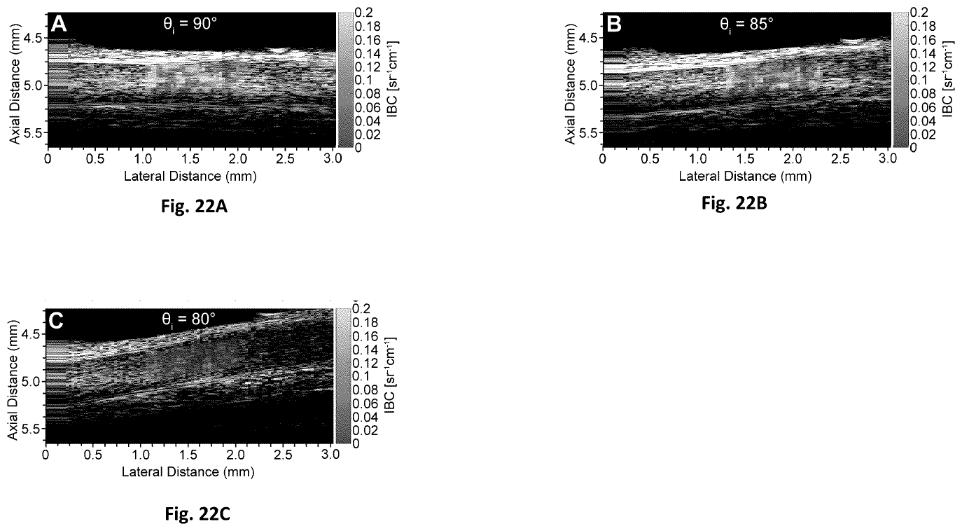

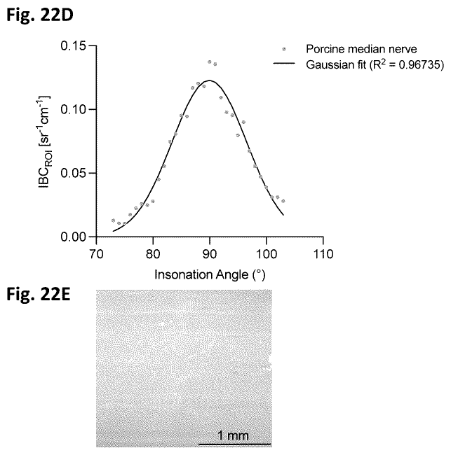

FIG. 22A-C show B-scan images and overlayed IBC parametric images for porcine median nerve at 3 representative angles of insonation (90, 85, 80 degrees). FIG. 22D presents the average IBC in a ROI as a function of all insonation angles (data points) and a Gaussian fit to the data (solid curve). Maximum IBC occurs near normal incidence and IBC magnitude decreases for angles above and below normal incidence. The IBC exhibited an angular dependence in nerve.

In summary, in this example, the capability of the quantitative ultrasound system and methods to detect neural tissue microstructure alignment was demonstrated. In porcine median nerve, the IBC was dependent on insonation angle, specifically, the magnitude of the IBC was greatest near normal incidence and decreased for angles greater than or less than normal incidence. The microstructural alignment observed in healthy nerve (FIG. 22E) can be disrupted by injury or pathology. Alterations in neural microstructure alignment will reduce the angular dependence of the IBC, illustrating the capability of the technique as a tool for non-invasively detecting and monitoring changes in neural tissue microstructure, as for example resulting from injury, disease, over-use, or malformation.

### Example 5: Quantitative Ultrasound System and Methods for Characterizing Tissue Microstructure of Ligament

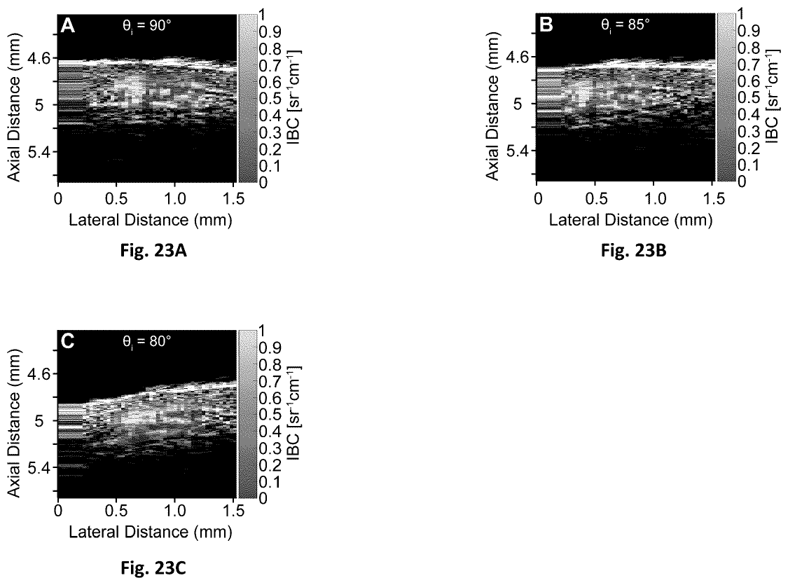

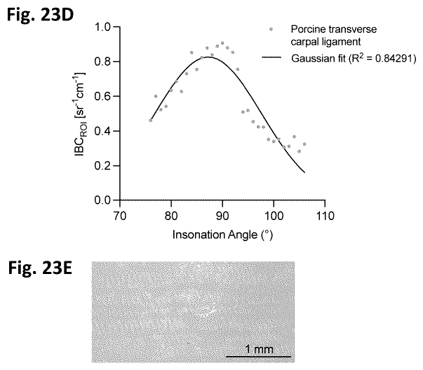

In the presented example, the quantitative ultrasound system, methods, and spectral analysis techniques developed in murine tendon were expanded for application to ligament. Briefly, the 58-MHz transducer system was used to acquire backscatter signals from porcine transverse carpal ligament ex vivo as a function of insonation angle. Backscatter data were then used to generate B-mode images, IBC metrics, and IBC parametric images as described previously. FIG. 23A-C show B-scan images and overlayed IBC parametric images for porcine transverse carpal ligament at 3 representative angles of insonation (90, 85, 80 degrees). FIG. 23D presents the average IBC in a ROI as a function of all insonation angles (data points) and a Gaussian fit to the data (solid curve). Maximum IBC occurs near normal incidence and IBC magnitude decreases for angles above and below normal incidence. Microstructural alignment in ligament is evident in microscopy image in FIG. 23E. Alterations in this ligament microstructure due to disease, injury, or malformation would result in a decrease in the angular dependence of the IBC and associated metrics. The IBC exhibited an angular dependence in ligament.

In summary, in this example, the capability of the quantitative ultrasound system and methods to detect microstructure alignment in ligament was demonstrated. In porcine transverse carpal ligament ex vivo, the IBC was dependent on insonation angle, where the magnitude of the IBC was greatest near normal incidence and decreased for angles greater than or less than normal incidence. Alterations in ligament microstructure will reduce the angular dependence of the IBC, illustrating the capability of the demonstrated technique as a tool for non-invasively detecting and monitoring changes in ligament tissue microstructure, as for example resulting from injury, disease, over-use, or malformation.

### Example 6: Estimation of Tissue Mechanical Properties Using Ultrasound Backscatter

A key challenge facing physical therapists, clinicians, and physicians is the development of patient-specific rehabilitation protocols that appropriately load tendon without inflicting further tendon damage during healing. The methods of this example noninvasively measure ultrasound backscatter metrics as predictors of tendon mechanical properties and monitor changes in tendon mechanical properties that may occur from disease, injury, aging, and healing. Thus, this example provides a nondestructive, noninvasive ultrasound imaging technology to quantitatively estimate clinically relevant, tendon mechanical properties to serve as a new tool to monitor tendon healing and guide patient-specific physical therapy.

Classically, destructive mechanical testing protocols are used to measure stress versus strain curves and estimate tissue mechanical properties. However, these classic techniques are not suitable for estimating mechanical properties in patients because the tissue is destroyed during the testing protocol. This example provides a quantitative ultrasound technique based on ultrasound backscatter to characterize tendon mechanical properties nondestructively and noninvasively.

Shown in FIG. 25, are experimental results that simultaneously measured stress versus strain (black curve) and ultrasound backscatter intensity versus strain (red curve) in porcine tendon. Stress and strain were measured using a standard tensile mechanical testing protocol and apparatus. A clinical, hand-held, point-of-care linear array ultrasound imaging device (Clarius L20) simultaneously measured ultrasound backscatter from the tendon during mechanical testing. Mechanical properties used to characterize tissues (FIG. 25) include transition strain (εₜ) and stress (σₜ), yield strain (ε_{y}) and stress (σ_{y}), ultimate strain (εᵤ) and stress (σᵤ), and Young's modulus (E). As FIG. 25 illustrates, the ultrasound backscatter intensity of the tendon varies with the applied load and strain. Data shown in FIG. 25 illustrate that metrics associated with ultrasound backscatter intensity as a function of strain can estimate clinically important mechanical properties. As examples, i) the inflection in ultrasound backscatter intensity (red star) can identify transition strain, ii) the slope of ultrasound backscatter intensity versus strain can provide a noninvasive surrogate measurement for Young's modulus, and iii) the end of the linear region of ultrasound backscatter intensity (green triangle) can provide an estimate of yield strain. Measurements of metrics associated with the angular dependence of the integrated backscatter coefficient may provide advanced metrics to serve as noninvasive surrogates for clinically relevant mechanical properties.

This technique can be valuable for physical therapists and clinicians, enabling them to design physical therapy regimens that safely load healing tendon while avoiding further damage to the tendon during rehabilitation protocols. Ultrasound backscatter intensity can be measured as tendon is loaded. Identification of the inflection in ultrasound backscatter intensity in real time (noted with the red star) would indicate to the clinician the transition strain, i.e, the transition from the toe region to the linear region. This transition is indicative of load before the linear elastic region and can serve as a safety metric to help prevent unintended injury to the tendon during physical therapy. As described above, additional metrics associated with measurements of ultrasound backscatter intensity as a function of tendon load (and/or strain) can also be identified to estimate linear elastic region Young's modulus and yield strain.

### REFERENCES

The following publications are incorporated herein by reference in their entirety.
1 Batista, F. et al. Achilles tendinopathy in diabetes mellitus. Foot Ankle Int 29, 498-501 (2008).
2 Boivin, G. P. et al. Biomechanical properties and histology of db/db diabetic mouse Achilles tendon. Muscles Ligaments Tendons J 4, 280-284 (2014).
3 Studentsova, V., Mora, K. M., Glasner, M. F., Buckley, M. R. & Loiselle, A. E. Obesity/Type II Diabetes Promotes Function-limiting Changes in Murine Tendons that are not reversed by Restoring Normal Metabolic Function. Sci Rep 8, 9218 (2018).
4 Guney, A. et al. Biomechanical properties of Achilles tendon in diabetic vs. non-diabetic patients. Exp Clin Endocrinol Diabetes 123, 428-432 (2015).
5 Lake, S. P., Miller, K. S., Elliott, D. M. & Soslowsky, L. J. Effect of fiber distribution and realignment on the nonlinear and inhomogeneous mechanical properties of human supraspinatus tendon under longitudinal tensile loading. J Orthop Res 27, 1596-1602 (2009).
6 Butler, D. L., Grood, E. S., Noyes, F. R., Zernicke, R. F. & Brackett, K. Effects of structure and strain measurement technique on the material properties of young human tendons and fascia. J Biomech 17, 579-596 (1984).
7 Federation, I. D. IDF Diabetes Atlas, (2021).
8 Ranger, T. A., Wong, A. M., Cook, J. L. & Gaida, J. E. Is there an association between tendinopathy and diabetes mellitus? A systematic review with meta-analysis. Br J Sports Med 50, 982-989 (2016).
9 David, M. A. et al. Tendon repair is compromised in a high fat diet-induced mouse model of obesity and type 2 diabetes. PLOS One 9, e91234 (2014).
10 Ackerman, J. E., Geary, M. B., Orner, C. A., Bawany, F. & Loiselle, A. E. Obesity/Type II diabetes alters macrophage polarization resulting in a fibrotic tendon healing response. PLOS One 12, e0181127 (2017).
11 Korcari, A., Przybelski, S. J., Gingery, A. & Loiselle, A. E. Impact of aging on tendon homeostasis, tendinopathy development, and impaired healing. Connect Tissue Res, 1-13 (2022).
12 Ackerman, J. E., Bah, I., Jonason, J. H., Buckley, M. R. & Loiselle, A. E. Aging does not alter tendon mechanical properties during homeostasis, but does impair flexor tendon healing. J Orthop Res (2017).
13 de Jonge, S. et al. Incidence of midportion Achilles tendinopathy in the general population. Br J Sports Med 45, 1026-1028 (2011).
14 Hullfish, T. J. & Baxter, J. R. A Reliable Method for Quantification of Tendon Structure Using B-Mode Ultrasound. J Ultrasound Med (2018).
15 Bashford, G. R., Tomsen, N., Arya, S., Burnfield, J. M. & Kulig, K. Tendinopathy discrimination by use of spatial frequency parameters in ultrasound B-mode images. IEEE Trans Med Imaging 27, 608-615 (2008).
16 Docking, S. I. & Cook, J. Pathological tendons maintain sufficient aligned fibrillar structure on ultrasound tissue characterization (UTC). Scand J Med Sci Sports 26, 675-683 (2016).
17 Duenwald-Kuehl, S., Chamberlain, C, Kobayashi, H, Lakes, R, Vanderby, R. Ultrasound analysis quantifies mechanical compromise in collagenase model for tendinopathy. ORS 2012 Annual Meeting, Poster No. 1288 (2012).
18 Hagan, K. L., Hullfish, T. J., Casey, E. & Baxter, J. R. Tendon Structure Quantified using Ultrasound Imaging Differs Based on Location and Training Type. J Appl Physiol (1985) (2018).
19 Kulig, K., Chang, Y. J., Winiarski, S. & Bashford, G. R. Ultrasound-Based Tendon Micromorphology Predicts Mechanical Characteristics of Degenerated Tendons. Ultrasound Med Biol 42, 664-673 (2016).
20 Tucker, J. J. et al. Effect of overuse-induced tendinopathy on tendon healing in a rat supraspinatus repair model. J Orthop Res 34, 161-166 (2016).
21 van Schie, H. T. M. et al. Ultrasonographic tissue characterisation of human Achilles tendons: quantification of tendon structure through a novel non-invasive approach. Brit J Sport Med 44, 1153-1159 (2010).
22 Abate, M., Salini, V., Antinolfi, P. & Schiavone, C. Ultrasound morphology of the Achilles in asymptomatic patients with and without diabetes. Foot Ankle Int 35, 44-49 (2014).
23 Akturk, M., Ozdemir, A., Maral, I., Yetkin, I. & Arslan, M. Evaluation of Achilles tendon thickening in type 2 diabetes mellitus. Exp Clin Endocrinol Diabetes 115, 92-96 (2007).
24 Couppe, C. et al. Human Achilles tendon glycation and function in diabetes. J Appl Physiol (1985) 120, 130-137 (2016).
25 de Jonge, S. et al. Achilles tendons in people with type 2 diabetes show mildly compromised structure: an ultrasound tissue characterisation study. Br J Sports Med 49, 995-999 (2015).
26 Crevier-Denoix, N. et al. Correlations between mean echogenicity and material properties of normal and diseased equine superficial digital flexor tendons: an in vitro segmental approach. J Biomech 38, 2212-2220 (2005).
27 Riggin, C. N., Sarver, J. J., Freedman, B. R., Thomas, S. J. & Soslowsky, L. J. Analysis of collagen organization in mouse achilles tendon using high-frequency ultrasound imaging. J Biomech Eng 136, 021029 (2014).
28 Tuthill, T. A., Rubin, J. M., Fowlkes, J. B., Jamadar, D. A. & Bude, R. O. Frequency analysis of echo texture in tendon. Ultrasound in Medicine and Biology 25, 959-968 (1999).
29 Buschmann, J. et al. Correspondence of high-frequency ultrasound and histomorphometry of healing rabbit Achilles tendon tissue. Connect Tissue Res 55, 123-131 (2014).
30 Chamberlain, C. S. et al. Temporal healing in rat achilles tendon: ultrasound correlations. Ann Biomed Eng 41, 477-487 (2013).
31 Freedman, B. R. et al. Nonsurgical treatment and early return to activity leads to improved Achilles tendon fatigue mechanics and functional outcomes during early healing in an animal model. J Orthop Res 34, 2172-2180 (2016).
32 Freedman, B. R. et al. Temporal Healing of Achilles Tendons After Injury in Rodents Depends on Surgical Treatment and Activity. J Am Acad Orthop Surg 25, 635-647 (2017).
33 Fryhofer, G. W. et al. Postinjury biomechanics of Achilles tendon vary by sex and hormone status. J Appl Physiol (1985) 121, 1106-1114 (2016).
34 Ghorayeb, S. R. et al. Ultrasonic assessment of extracellular matrix content in healing Achilles tendon. IEEE Trans Ultrason Ferroelectr Freq Control 59, 694-702 (2012).
35 Duenwald-Kuehl, S., Lakes, R. & Vanderby, R., Jr. Strain-induced damage reduces echo intensity changes in tendon during loading. J Biomech 45, 1607-1611 (2012).
36 Ackerman, J. S., Valentina & Loiselle, Alayna. Non-invasive ultrasound quantification of Scar Tissue Volume predicts functional changes during tendon healing. ((2018)).
37 Duenwald, S., Kobayashi, H., Frisch, K., Lakes, R. & Vanderby, R., Jr. Ultrasound echo is related to stress and strain in tendon. J Biomech 44, 424-429 (2011).
38 Duenwald-Kuehl, S., Kobayashi, H., Lakes, R. & Vanderby, R., Jr. Time-dependent ultrasound echo changes occur in tendon during viscoelastic testing. J Biomech Eng 134, 111006 (2012).
39 Frisch, K. E., Marcu, D., Baer, G. S., Thelen, D. G. & Vanderby, R. Influence of tendon tears on ultrasound echo intensity in response to loading. J Biomech 47, 3813-3819 (2014).
40 Lee, S. Y. et al. Characteristics of Sonography in a Rat Achilles Tendinopathy Model: Possible Non-invasive Predictors of Biomechanics. Sci Rep 7, 5100 (2017).
41 Suydam, S. M. & Buchanan, T. S. Is echogenicity a viable metric for evaluating tendon properties in vivo? J Biomech 47, 1806-1809 (2014).
42 Cobbold, R. S. C. in Foundations of Biomedical Ultrasound Ch. 7, 414-422 (Oxford University Press, 2007).
43 Szabo, T. L. in Diagnostic Ultrasound Imaging: Inside Out Ch. 10, 387-388 (Elsevier Inc. Academic Press, 2014).
44 Szabo, T. L. in Diagnostic Ultrasound Imaging: Inside Out Ch. 1, 26 (Elsevier Inc. Academic Press, 2014).
45 Oelze, M. L. & Mamou, J. Review of Quantitative Ultrasound: Envelope Statistics and Backscatter Coefficient Imaging and Contributions to Diagnostic Ultrasound. IEEE Trans Ultrason Ferroelectr Freq Control 63, 336-351 (2016).
46 Cloutier, G., Destrempes, F., Yu, F. & Tang, A. Quantitative ultrasound imaging of soft biological tissues: a primer for radiologists and medical physicists. Insights Imaging 12, 127 (2021).
47 Dalecki, D., Mercado, K. P. & Hocking, D. C. Quantitative Ultrasound for Nondestructive Characterization of Engineered Tissues and Biomaterials. Ann Biomed Eng 44, 636-648 (2016).
48 Mercado, K. P. Developing High-Frequency Quantitative Ultrasound Techniques to Characterize Three-Dimensional Engineered Tissues PhD thesis, University of Rochester, (2015).
49 Mercado, K. P., Helguera, M., Hocking, D. C. & Dalecki, D. Estimating cell concentration in three-dimensional engineered tissues using high frequency quantitative ultrasound. Ann Biomed Eng 42, 1292-1304 (2014).
50 Mercado, K. P., Helguera, M., Hocking, D. C. & Dalecki, D. Noninvasive Quantitative Imaging of Collagen Microstructure in Three-Dimensional Hydrogels Using High-Frequency Ultrasound. Tissue Eng Part C Methods 21, 671-682 (2015).
51 Cobbold, R. S. C. in Foundations of Biomedical Ultrasound Ch. 5, 270-271 (Oxford University Press, 2007).
52 Insana, M. F. & Hall, T. J. Parametric ultrasound imaging from backscatter coefficient measurements: image formation and interpretation. Ultrason Imaging 12, 245-267 (1990).
53 Mottley, J. G. & Miller, J. G. Anisotropy of the ultrasonic backscatter of myocardial tissue: I. Theory and measurements in vitro. J Acoust Soc Am 83, 755-761 (1988).
54 Hoffmeister, B. K., Wong, A. K., Verdonk, E. D., Wickline, S. A. & Miller, J. G. Comparison of the anisotropy of apparent integrated ultrasonic backscatter from fixed human tendon and fixed human myocardium. J Acoust Soc Am 97, 1307-1313 (1995).
55 Holland, M. R. et al. Effects of myocardial fiber orientation in echocardiography: quantitative measurements and computer simulation of the regional dependence of backscattered ultrasound in the parasternal short-axis view. J Am Soc Echocardiogr 11, 929-937 (1998).
56 Madaras, E. I., Perez, J., Sobel, B. E., Mottley, J. G. & Miller, J. G. Anisotropy of the ultrasonic backscatter of myocardial tissue: II. Measurements in vivo. J Acoust Soc Am 83, 762-769 (1988).
57 Hall, C. S., Scott, M. J., Lanza, G. M., Miller, J. G. & Wickline, S. A. The extracellular matrix is an important source of ultrasound backscatter from myocardium. J Acoust Soc Am 107, 612-619 (2000).
58 Wickline, S. A., Verdonk, E. D., Wong, A. K., Shepard, R. K. & Miller, J. G. Structural remodeling of human myocardial tissue after infarction. Quantification with ultrasonic backscatter. Circulation 85, 259-268 (1992).
59 Holland, M. R. et al. Comparison of integrated backscatter values obtained with acoustic densitometry with values derived from spectral analysis of digitized signals from a clinical imaging system. J Am Soc Echocardiogr 10, 511-517 (1997).
60 Hete, B. & Shung, K. K. Scattering of ultrasound from skeletal muscle tissue. IEEE Trans Ultrason Ferroelectr Freq Control 40, 354-365 (1993).
61 Garcia, T., Hornof, W. J. & Insana, M. F. On the ultrasonic properties of tendon. Ultrasound Med Biol 29, 1787-1797 (2003).
62 Szabo, T. L. in Diagnostic Ultrasound Imaging: Inside Out Ch. 6, 178-181 (Elsevier Inc. Academic Press, 2014).
63 Libgot-Calle, R., Ossant, F., Gruel, Y., Lermusiaux, P. & Patat, F. High frequency ultrasound device to investigate the acoustic properties of whole blood during coagulation. Ultrasound Med Biol 34, 252-264 (2008).
64 Ganzalez, R. C., Woods, R. E. in Digital Image Processing Ch. 4, 175-177 (Prentice-Hall, Inc., 2002).
65 Parker, K. J. Ultrasonic attenuation and absorption in liver tissue. Ultrasound Med Biol 9, 363-369 (1983).
66 Moran, C. M., Bush, N. L. & Bamber, J. C. Ultrasonic propagation properties of excised human skin. Ultrasound Med Biol 21, 1177-1190 (1995).
67 Pinkerton, J. M. M. The absorption of ultrasonic waves in liquids and its relation to molecular constitution. Proceedings of the Physical Society. Section B 62 (1949).
68 Szabo, T. L. in Diagnostic Ultrasound Imaging: Inside Out Ch. 9, 296-299 (Elsevier Inc. Academic Press, 2014).
69 Voleti, P. B., Buckley, M. R. & Soslowsky, L. J. Tendon healing: repair and regeneration. Annu Rev Biomed Eng 14, 47-71 (2012).
70 Afolabi, B. I., Idowu, B. M. & Onigbinde, S. O. Achilles tendon degeneration on ultrasound in type 2 diabetic patients. J Ultrason 20, e291-e299 (2021).
71 Connizzo, B. K., Sarver, J. J., Birk, D. E., Soslowsky, L. J. & Iozzo, R. V. Effect of age and proteoglycan deficiency on collagen fiber re-alignment and mechanical properties in mouse supraspinatus tendon. J Biomech Eng 135, 021019 (2013).
72 Zuskov, A. et al. Tendon Biomechanics and Crimp Properties Following Fatigue Loading Are Influenced by Tendon Type and Age in Mice. J Orthop Res 38, 36-42 (2020).
73 Wang, P. H., Luh, J. J., Chen, W. S. & Li, M. L. In vivo photoacoustic micro-imaging of microvascular changes for Achilles tendon injury on a mouse model. Biomed Opt Express 2, 1462-1469 (2011).
74 Ackerman, J. E. & Loiselle, A. E. Murine Flexor Tendon Injury and Repair Surgery. J Vis Exp (2016).
75 Rollins M D, et al. J Pediatr. 2022 January; 240:122-128.

1. A method of characterizing a tissue, comprising:
providing a set of at least one ultrasound transducers and a set of at least one ultrasound receivers;
insonating the tissue with one of the set of ultrasound transducers from a first position;
collecting a first reflected ultrasound energy with at least one of the set of ultrasound receivers;
insonating the tissue with one of the set of ultrasound transducers from a second position;
collecting a second reflected ultrasound energy with at least one of the set of ultrasound receivers;
calculating a first and second average integrated backscatter coefficient of the tissue from the first and second reflected ultrasound energies respectively; and
determining at least one characteristic of the tissue based on the first and second integrated backscatter coefficients.

2. The method of claim 1, wherein the at least one ultrasound transducer of the set is selected from the group of: a single-element transducer, a multi-element transducer, an array transducer, a 1-D array transducer, a 1.5-D array transducer, a 2-D array transducer, a ring array transducer, and a linear array transducer.

3. The method of claim 1, wherein the tissue is selected from the group of: a tendon, a ligament, a cartilage, a nerve, a heart, a cornea, a skeletal muscle, a smooth muscle, connective tissues, liver, skin, extracellular matrices, or an artificially fabricated tissue.

4. The method of claim 1, further comprising the step of positioning the transducer automatically.

5. The method of claim 1, wherein the reflected ultrasound energies comprise a backscattered echo.

6. The method of claim 1, wherein at least one of the first position and second position comprise an insonation angle of about 90° relative to the tissue surface.

7. The method of claim 1, further comprising the steps of:
insonating the tissue with one of the set of ultrasound transducers from a third position;
collecting a third reflected ultrasound energy with at least one of the set of ultrasound receivers; and
calculating a third average integrated backscatter coefficient of the tissue from the third reflected ultrasound energy,
wherein the first position comprises an insonation angle less than 90° relative to the tissue surface, the second position comprises an insonation angle of about 90° relative to the tissue surface, and the third position comprises an insonation angle of greater than 90° relative to the tissue surface,
and wherein determining the at least one characteristic of the tissue is further based on third average integrated backscatter coefficient.

8. The method of claim 6, further comprising the step of quantifying a relationship between the average integrated backscatter coefficient and the insonation angle of the position.

9. The method of claim 8, wherein quantifying the relationship between the average integrated backscatter coefficient and the insonation angle comprises determining a Gaussian fit of the average integrated backscatter coefficient and the insonation angle.

10. (canceled)

11. The method of claim 8, wherein quantifying the relationship between estimated integrated backscatter coefficient and the insonation angle further comprises calculating a metric of the group selected from: the maximum average integrated backscatter coefficient value of the Gaussian fit, the change in amplitude of the Gaussian fit between the maximum value and the value corresponding with any other insonation angle, the linear rate of change of the derivative of the Gaussian fit, and the difference between the first and second average backscatter coefficients.

12. The method of claim 11, wherein the metric is the change in amplitude of the Gaussian fit between the maximum value and the value corresponding with any other insonation angle, and wherein the any other insonation angle is within the range of about 70° to about 110° relative to the tissue surface.

13. The method of claim 1, wherein the at least one characteristic of the tissue is a property of the tissue structure.

14. The method of claim 13, wherein the property of the tissue structure is one or more of the group selected from: tissue microstructure, extracellular matrix fiber organization, extracellular matrix fiber alignment, collagen fiber organization, collagen fiber alignment, extracellular matrix fiber crosslinking, collagen fiber crosslinking, distribution of extracellular matrix fiber subtypes, distribution of collagen fiber subtypes, extracellular matrix fiber density, collagen fiber density, extracellular matrix fiber length, collagen fiber length, cellular alignment, cellular organization, vascular alignment, and vascular organization.

15. The method of claim 1, wherein the at least one characteristic of the tissue is a material property of the tissue.

16. The method of claim 15, wherein the material property is one or more of the group selected from: stiffness, Young's modulus, transition strain, transition stress, yield strain, yield stress, ultimate strain, ultimate stress, compressive load, tensional load, and viscoelasticity.

17. The method of claim 1, wherein the at least one characteristic of the tissue comprises one or more of the group selected from the following: age, inflammation, vascularization, scarring, fibrosis, and steatosis.

18. The method of claim 1, further comprising the step of diagnosing a subject based on the determination of the at least one characteristic of the tissue.

19. The method of claim 18, wherein the diagnosis comprises one or more of the group selected from: assigning a risk of tissue injury, diagnosing a subject with an autoimmune disease, diagnosing a subject with a metabolic disease, and determining the healing of an injured tissue.

20. The method of claim 1, further comprising one or more of the steps selected from the group consisting of: determining a treatment for the subject based on the determination of the at least one characteristic of the tissue and determining a rehabilitation therapy for the subject based on the determination of the at least one characteristic of the tissue.

21. A system for characterizing a tissue, comprising:
an imaging device comprising a set of at least one ultrasound transducers and a set of at least one ultrasound receivers;
a display;
a processor communicatively connected to the imaging device and the display; and
a non-transitory computer readable medium with instructions stored thereon, which when executed by a processor perform steps comprising:
insonating the tissue with one of the set of ultrasound transducers from a first position;
collecting a first reflected ultrasound energy with at least one of the set of ultrasound receivers;
insonating the tissue with one of the set of ultrasound transducers from a second position;
collecting a second reflected ultrasound energy with at least one of the set of ultrasound receivers;
calculating a first and second average integrated backscatter coefficient of the tissue from the first and second reflected ultrasound energies respectively; and
determining at least one characteristic of the tissue based on the first and second integrated backscatter coefficients.

22-39. (canceled)
